(19) United States
(12) Patent Application Publication
(10) Pub. No.: US 2025/0276996 A1
(43) Pub. Date: Sep. 4, 2025

(54) CATIONICALLY-ENFRAMED HIGH DENSITY AROMATIC PEPTIDES

(71) Applicant: Research Foundation of the City University of New York, New York, NY (US)

(72) Inventors: Alexander Birk, Bellerose, NY (US); Margaret MacNeil, Scarsdale, NY (US)

(21) Appl. No.: 19/205,418

(22) Filed: May 12, 2025

Related U.S. Application Data
(63) Continuation-in-part of application No. PCT/US23/79362, filed on Nov. 10, 2023.
(60) Provisional application No. 63/383,129, filed on Nov. 10, 2022.

(51) Int. Cl. C07K 5/11; A61K 38/00; A61K 47/54; A61P 27/02

(52) U.S. Cl. CPC C07K 5/1019; A61K 47/557; A61P 27/02; A61K 38/00

(57) ABSTRACT

A method of treating mitochondria dysfunction-associated discloses by administering a biotinylated peptide with r residues (from 4-10) that are either cationic or aromatic. The peptide has a cationic D-amino acid at the N-terminus and a cationic D-amino acid at the C-terminus. The net number of positive residues (np), is 2≤np≤r−2. The net number of the aromatic residues is greater than or equal to the net number of positive residues.

### CROSS-REFERENCE TO RELATED APPLICATIONS

This application claims priority to, and is a continuation-in-part of, international patent application PCT/US2023/079362 (filed Nov. 10, 2023) which is a non-provisional of U.S. Patent Application 63/383,129 (Nov. 10, 2022), the entirety of which are incorporated herein by reference.

### REFERENCE TO A SEQUENCE LISTING

This application contains a Sequence Listing in computer readable form. The computer readable form is incorporated herein by reference. The computer readable file is named Sequence.xml and was created on Nov. 8, 2023 (38 KB).

### BACKGROUND OF THE INVENTION

Mitochondrial dysfunction contributes to many debilitating disorders, including, but not limited to, heart attack, stroke, heart failure, muscle weakness and dystrophy/atrophy, Alzheimer's and Parkinson's diseases, glaucoma, acute and chronic kidney failure, acute and chronic liver failure, diabetes, and cancer. Novel mitochondrial membrane-stabilizing analogs are of particular clinical importance to treat this multitude of acute and chronic mitochondrial dysfunctions, which afflict a large proportion of the human population at some stage during their lifetime.

The mitochondrial membrane potential (ΔΨm) is created by the accumulation of protons on an outer leaflet of the inner mitochondrial membrane and drives the synthesis of most cellular ATP, which is essential for cellular bioenergetics and survival. The ΔΨm also facilitates the electrogenic transport of cations, such as Ca²⁺, and regulates generation of reactive oxygen species, which serves as a powerful bioenergetic and stress-signaling regulator. Proton trapping on the outer leaflet of the inner mitochondrial membrane of mitochondrial cristae could be controlled by cardiolipin (CL) when the local pH is above 8. However, there is presently no technology that effectively targets strong bases to cardiolipin.

While some mitochondrial affecting compounds are available, it would be desirable to provide additional compounds so as to broaden the available options.

The discussion above is merely provided for general background information and is not intended to be used as an aid in determining the scope of the claimed subject matter.

### SUMMARY

This disclosure provides a peptide with r residues (from 4-10) that are either cationic or aromatic. The peptide has a cationic D-amino acid at the N-terminus and a cationic D-amino acid at the C-terminus. The net number of positive residues (np), is 2≤np≤r−2. The net number of the aromatic residues is greater than or equal to the net number of positive residues.

An advantage that may be realized in the practice of some disclosed embodiments is that the peptides are useful to treat mitochondria dysfunction-associated diseases.

In a first embodiment, a method of treating an eye of a patient is provided. The method comprises administering to the eye a composition comprising biotin covalently bound to a peptide of dArg-Phe-Phe-dArg (SEQ ID NO: 1).

In a second embodiment, a method of treating an eye of a patient is provided. The method comprises administering to the eye a composition comprising: an N-terminus and a C-terminus; r amino acid residues, wherein 4≤r≤10 and the amino acid residues are either cationic amino acid residues or aromatic amino acid residues, the r amino acid residues including (1) a cationic D-amino acid (dC) at the N-terminus and (2) a cationic D-amino acid (mdC) at the C-terminus; wherein a net number of positive residues (np), including the cationic D-amino acid (dC) and the cationic D-amino acid (mdC), is 2≤np≤r−2; and wherein a net number of the aromatic amino acid residues (na) is na≥np, and the aromatic amino acid residues include at least two aromatic amino acids that are sequentially positioned in the peptide.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a fluorescent microscopy image showing MitoTracker (upper panels, MitoT) used to detect cells in a monolayer on a 6 well plate. StreptAvidin (bottom panels, StreptAv) was used to detect biotin-HDAP2 in the same cells. Although MitoTracker labeled both the control and biotin-HDAP2 treated cells, StreptAvidin labeled only biotin-HDAP2 treated cells. The white oval identifies three such cells double-labeled with MitoT and StrepAvidin.

FIG. 2 is a fluorescent microscopy image showing MitoTracker (Red) co-localizes with Streptavidin, ALEXA FLUOR™ 488 (green) as indicated by appearance of yellow inside of the cell. Bright red aggregates are indicative of mitochondria aggregation/mitophogy and are not labeled by Streptavidin.

FIG. 3A shows a representative fluorescence emission spectra of NAO (1 μM) in the presence of different phospholipids (POPC and CL) alone and with biotin-HDAP2 (ex 495 nm).

FIG. 3B shows a quantitative analysis of plot A demonstrates that biotin-HDAP2 displaces nonyl acridine orange (NAO) interaction with CL.

FIG. 3C shows a representative of fluorescence microscopy of NAO (1 μM) in MDBK cells in the absence or presence of biotin-HDAP2 (10 μM) (ex 579 nm).

FIG. 3D shows a representative fluorescence emission spectra of Nile Red (1 μM) in the presence of different phospholipids (POPC and CL) alone and with biotin-HDAP2 (ex 540 nm). Right vertical line is at the λₘₐₓ of NR for CL. Left vertical line is at the λₘₐₓ of NR for CL in the presence of biotin-HDAP2. Middle vertical line is at the λₘₐₓ of NR for POPC alone and in the presence of biotin-HDAP2.

FIG. 4 shows cells grown for 5-7 days showed monolayer disruption, cell detachment, and death, which could be easily detected by weak methylene blue staining. In the presence of biotin-HDAP2, cells remained attached to the plate and cell monolayer appeared to be intact as demonstrated by strong methylene blue staining. Samples were analyzed and counted as described in methodology. Error bars indicate standard error for each time point and specified condition (n=5).

FIG. 5 shows cells grown for 5-7 days were stained with MitoTracker-Red to detect mitochondrial membrane potential and DAPI to detect nuclei and cell location. Biotin-HDAP2 improved mitochondrial membrane potential compared to control cell, as evidenced by the brighter MitoTracker staining.

FIG. 6 shows cells grown in serum-free media for 5-7 days, with or without analog biotin-HDAP2, were stained with DAPI to detect nuclei and the location of cells and the cellular oxidative marker, CM-H2DCFFDA to detect cellular oxidative stress. Cell starvation increased CM-H2DCFFDA staining and oxidative stress, indicated by the higher level of fluorescence compared to the staining observed in cells treated with biotin-HDAP2. Biotin-HDAP2 improved mitochondrial membrane potential compared to control cells.

FIG. 7A depicts retinal cryostat sections that were incubated with strepavidin-conjugated AlexaFluor 488 to reveal biotinylated HDAP2 or endogenous biotin (green), ToPro 3 to label cell nuclei (blue), and imaged in the confocal microscope using the same PMT voltage to show the distribution of labeled biotin-HDAP2. Control sections showed little to no labeling with streptavidin (green), while the biotin-HDAP2 exposed animals showed labeling throughout all layers of the retina, including retinal ganglion cell (RGC) somas and axons. The distribution of biotin-HDAP2 was similar to the distribution of Mitotracker and CoxIV, which label mitochondria and also overlaps glutamine synthetase staining, which labels Müller cells. GC, ganglion cell layer; IPL, inner plexiform layer; INL, inner nuclear layer; OPL, outer plexiform layer; ONL, outer nuclear layer, IS, inner segments of photoreceptors.

FIG. 7B depicts wholemounted retina labeled with glutamine synthetase and biotin-HDAP2 to show the significant accumulation of biotin-HDAP2 in retinal Müller cells. FIG. 7B shows a triple labeled wholemount with ToPro-stained nuclei in blue, biotin-HDAP2 colocalized with glutamine synthetase (gray), and a high density of biotin-HDAP2 labeling alongside blood vessels (red).

FIG. 8 illustrates retinal explants labeled with Brn3a (green) to label retinal ganglion cells (RGCs), ToPro3 to label all cell nuclei (red) and ChAT to label displaced amacrine cells (blue). Explants treated with HDAPs showed more labeled RGCs (green) compared to untreated controls indicating protection by the peptide.

FIG. 9 depicts retinal wholemounts that were stained for retinal ganglion cells (mouse anti-Rbpms, green) and starburst amacrine cells (goat anti-ChAT, magenta) following optic nerve crush (ONC). The numbers of ChAT-labeled cells were unchanged in all conditions, but ONC induced significant death of retina ganglion cells. However, the survival of retinal ganglion cells in biotin-HDAP2-treated animals was 50% greater compared to untreated controls both when biotin-HDAP2 was administered either via intraperitoneal (***p<0.0001) or subcutaneous (**p<0.001) administration.

FIG. 10A depict Madin-Darby Bovine Kidney (MDBK) cultured cells incubated with DAPI and streptavidin AF 488 to demonstrate that biotinylated biotin-HDAP2 readily loads into cells and is expressed at levels far exceeding (>10×) endogenous biotin.

FIG. 10B is a graph comparing the biotinylated biotin-HDAP2 to endogenous biotin.

FIG. 10C depicts MDBK cells labeled with streptavidin AF488 to identify biotin-HDAP2 (green) and MitoTracker Red CMXRos (magenta) to label mitochondria. Co-localization is indicated by the yellow-gray labeling.

FIG. 10D demonstrates biotin-HDAP2 improves mitochondrial potential by 2.5-fold.

FIG. 10E shows biotin-HDAP2 decreases fluorescence of CM-H2-DCFDA by 80% compared to control, demonstrating decreases in oxidative stress.

FIG. 10F illustrates biotin-HDAP2 increased AlamarBlue fluorescence by 50% compared to control, demonstrating that biotin-HDAP2 increased cell survival. Differences between Control and biotin-HDAP2-treated conditions were all significantly different from each other (n=5 for each assay; **P<0.01), ***P<0.001, and ****P<0.0001).

FIG. 11A shows the ratios of RGCs labeled with RBPMS (green) and starburst amacrine cells labeled with ChAT (magenta) were used to control for differences in cell density across the retina and to standardize the densities following optic nerve crush (ONC). After ONC, the numbers of RBPMS-labeled neurons decreased significantly, but the distribution of ChAT cells was unaffected.

FIG. 11B shows the ratios of RBPMS to ChAT was about 4 to 1 in central retina and about 3 to 1 elsewhere.

FIG. 11C shows the density of ChAT labeled cells in Control, ONC and biotin-HDAP2 treatment groups in central, midperipheral and peripheral eccentricities were not significantly different from each other (P>0.05, n=11-15 samples per eccentricity), demonstrating their usefulness as an internal control.

FIG. 12 depicts retinal wholemounts were stained for RGCs (RBPMS, green) and starburst amacrine cells (ChAT, magenta) following optic nerve crush (ONC). The numbers of ChAT-labeled cells were unchanged in all conditions, but ONC induced significant death of RGCs. However, cell survival in biotin-HDAP2 treated animals was 40% greater than observed in untreated controls.

FIG. 13A shows that, following ONC, the ratio of RBPMS to ChAT decreased by 80% compared to unoperated control retinas (ANOVA, P<0.001).

FIG. 13B shows expression of RBPMS doubled in biotin-HDAP2 treated animals compared to untreated controls (ANOVA, P<0.001).

FIG. 13C shows survival of RGCs was significantly greater in biotin-HDAP2 treated animals than for the untreated controls at all retinal locations (Student's t-tests, ****P<0.0001, ***P<0.001).

FIG. 14 shows biotin-HDAP2 decreases the intensity of NAO fluorescence with POPC-CL liposomes in a dose-dependent manner.

FIG. 15A shows detection of POPc-biotin-HDAP2 (middle panel) and CL-biotin-HDAP2 vesicles on the surface of the cell culture plates-treated with 100 nM NR.

FIG. 15B shows intensity of NR labeling with biotin-HDAP2-POPC and biotin-HDAP2-CL vesicles. Error bars represent SEM (n=6) **P<0.01.

FIG. 15C are representative images of CL-biotin-HDAP2 vesicles labeled with 100 nM of TMRM alone (left panel) and in the presence of proton gradient uncouplers 100 μM of CCCP (middle panel) and 1 mM DNP (right panel).

FIG. 15D shows the results of quantitative analysis of TMRM-dependent labeling of CL-biotin-HDAP2 vesicles in the presence of CCCP and DNP. Error bars represent SEM (n=6) ***P<0.001.

FIG. 16A depicts representative images of MDBK cells incubated with and streptavidin AlexaFluor 488 to demonstrate that 10 μM of N-biotinylated biotin-HDAP2 readily loads into cells (right panel), compared with 10 μM of biocytin (middle panel).

FIG. 16B shows the results of quantitative analysis of biotin-HDAP2 and biocalytin uptake in MDBK cells. Error bars represent SEM (n=5) ***P<0.001.

FIG. 16C are images of cultured MDBK cells labeled with streptavidin AF488 to identify biotin-HDAP2 (green) and Mitotracker Red CMXRos (red) to label mitochondria. Co-localization is indicated by the yellow labeling.

FIG. 16D is a graph showing biotin-HDAP2 promotes survival of serum-starved MDBK cells in a dose-dependent manner, with EC50 of 100 nM.

FIG. 17A shows an image of ARPE-19 cells incubated with TMRM to demonstrate changes in the mitochondrial membrane potential (red).

FIG. 17B shows that, when cells were incubated with 10 μM CCCP, TMRM staining was virtually extinguished.

FIG. 17C shows increased fluorescence when 1 μM biotin-HDAP2 was added in addition to 10 μM CCCP.

FIG. 17D is a graph showing percentage change for the images in FIGS. 17A-C. FIG. 17D shows biotin-HDAP2 prevented CCCP-mediated decreases in the mitochondrial membrane potential in a dose-dependent manner. Error bars represent SEM (n=5) ***P<0.001.

FIG. 18A shows the results of quantitative analysis of the effect of biotin-HDAP2 and HDAP6 (SEQ ID NO: 10) on the restoration of mitochondrial membrane potential in ARPE-19 cells.

FIG. 18B depicts images of HK-2 cells that were serum starved for 3 days in the presence or absence of biotin-HDAP2, biotin-HDAP4, and biotin-HDAP6 (SEQ ID NO: 13).

FIG. 19 depicts images of mitochondria in optic nerve axons from Control (left), Untreated (middle), and biotin-HDAP2-treated (right) animals with cumulative intraocular pressures (cIOPs) noted. Mitochondria in biotin-HDAP2-treated nerves appeared similar in shape and density to those in control nerves and contained many cristae. Untreated axons had few mitochondria, increased mitophagy, disrupted myelin sheaths and opaque axoplasm. Scale=500 nm.

FIG. 20 shows semithin cross-sections of optic nerves from Control, Untreated, and biotin-HDAP2-treated animals. Despite similarly elevated cIOPs in the Untreated and biotin-HDAP2-treated groups, only the biotin-HDAP2-treated nerves exhibited preserved axon morphology. Scale=20 μm.

FIG. 21 shows whole-mounted retinas stained for RGCs using rabbit anti-RBPMS (green). Insets show high magnification labeling. Untreated animals with cIOPs>45 mmHg lost about 90% of RGCs (middle), while biotin-HDAP2-treated retinas (right), had RGC counts similar to controls (left). Scale=100 μm.

FIG. 22 is a graph showing linear regression of RGC counts as a function of cIOP. Each point represents one retina. Solid lines show best-fit linear regressions for each group. Slopes did not differ significantly (p=0.64), but overall RGC counts were significantly higher in the biotin-HDAP2 group (p<0.001).

FIG. 23 is a graph showing biotin-free HDAP2 does not protect cells during serum starvation.

FIG. 24 is a graph showing topical antimycin-induced scratching in mice is inhibited by topical biotin-HDAP2 and potentiated by topical biotin-free HDAP2.

### DETAILED DESCRIPTION OF THE INVENTION

This disclosure provides compounds that treat mitochondria dysfunction-associated diseases. The compounds act by binding to cardiolipin (CL) and stabilizing mitochondrial membrane potential and preventing cellular oxidative stress. The chemical nature of these compounds allows for rapid organization of mitochondrial cardiolipin-containing membranes to improve electron transfer and electron/energy storage for optimizing of mitochondrial functions and cellular bioenergetics. Examples of mitochondria dysfunction-associated diseases include optic nerve injury and axonal injury of retina ganglion cells, including traumatic optic nerve injury and glaucoma.

The disclosed compounds may be administered using a variety of means. For example, the compositions may be administered directly to an eye (including a retina) of a patient as, for example, with eyedrops or by direct ocular injection. The eyedrops may include additional additives such as buffers, stabilizers and the like. The compositions may also be indirectly administered to the patient's eye by administering the composition intravenously to intraperitoneally. As discussed elsewhere in this specification, the compounds localize within mitochondria-dense regions of the retina. In one embodiment, the patient is a human patient.

The administration may be repeated at regular time intervals. For example, in some embodiments, the administering occurs once per day for a period of at least three months. In another embodiment, the administering occurs twice per day for a period of at least three months.

The disclosed compounds are biotinylated high-density aromatic peptides that bind selectively to cardiolipin-containing membranes, by selectively interacting with phosphate groups of cardiolipin and supporting structural conformation of cardiolipin. Furthermore, binding of certain biotinylated high-density aromatic peptides to phosphate groups of cardiolipin will generate more alkaline conditions in the polar part of cardiolipin, which would be very beneficial for cardiolipin-mediated proton trapping, and stabilization of mitochondrial membrane potential and ATP synthesis.

These high-density aromatic peptides comprise diaromatic or triaromatic peptides (e.g Phe, Tyr, Trp) enframed by least two net positive charges (e.g. Arg, His, Lys) and has between four and about ten amino acids and is covalently bound at the N terminal end. In one embodiment, the number of amino acids present in the peptides is between 4 and 10, between 4 and 8, between 4 and 6 or 4. In one embodiment, each aromatic residue in the peptide is adjacent to at least one other aromatic residue (e.g. the aromatic residues appear in adjacent pairs or adjacent groups of three). A relationship exists between the maximum number of net positive charges (np) at physiological pH and the total number of amino acid residues (r). The total number of net positive amino acids is at least two and is less than or equal to r−2. A relationship exists between the minimum number of net aromatic groups (na) and the total number of net positive charges (np). The number of net aromatic groups (na) is more than or equal to np.

The amino acids may be naturally occurring. Naturally occurring amino acids include, for example, the twenty most common levorotatory (L) amino acids normally found in mammalian proteins (i.e., alanine (Ala), arginine (Arg), asparagine (Asn), aspartic acid (Asp), cysteine (Cys), glutamine (Glu), glutamic acid (Glu), glycine (Gly), histidine (His), isoleucine (Ileu), leucine (Leu), lysine (Lys), methionine (Met), phenylalanine (Phe), proline (Pro), serine (Ser), threonine (Thr), tryptophan, (Trp), tyrosine (Tyr), and valine (Val)). Other naturally occurring amino acids include amino acids that are synthesized in metabolic processes not associated with protein synthesis. For example, the amino acids ornithine and citrulline are synthesized in mammalian metabolism during the production of urea. Naturally occurring amino acids also include derivatives of naturally occurring amino acids. The derivatives of naturally occurring amino acids may, for example, include the addition of one or more chemical groups to the naturally occurring amino acid.

One or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. The group can be any chemical group that can be added to an aromatic ring. Some examples of such groups include branched or unbranched C₁-C₄ alkyl, such as methyl, ethyl, n-propyl, isopropyl, butyl, isobutyl, or t-butyl, C₁-C₄ alkyloxy (i.e., alkoxy), amino, C₁-C₄ alkylamino and C₁-C₄ dialkylamino (e.g., methylamino, dimethylamino), nitro, hydroxyl, halo (i.e., fluoro, chloro, bromo, or iodo). Some specific examples of non-naturally occurring derivatives of naturally occurring amino acids include norvaline (Nva), norleucine (Nle), and hydroxyproline (Hyp).

Another example of a modification of an amino acid in a peptide is the derivatization of a carboxyl group of an aspartic acid or a glutamic acid residue of the peptide. One example of derivatization is amidation with ammonia or with a primary or secondary amine, e.g. methylamine, ethylamine, dimethylamine or diethylamine. Another example of derivatization includes esterification with, for example, methyl or ethyl alcohol.

The peptides can contain one or more non-naturally occurring amino acids. The non-naturally occurring amino acids may be L-, dextrorotatory (D), or mixtures thereof. In one embodiment, the peptide has no amino acids that are naturally occurring or are recognized by common proteases. The non-naturally occurring amino acid can be present at any position in the peptide. For example, the non-naturally occurring amino acid can be at the N-terminus, the C-terminus, or at any position between the N-terminus and the C-terminus. The non-natural amino acids may, for example, comprise alkyl, aryl, or alkylaryl groups. Some examples of alkyl amino acids include α-aminobutyric acid, β-aminobutyric acid, γ-aminobutyric acid, δ-aminovaleric acid, and c-aminocaproic acid. Some examples of aryl amino acids include ortho-, meta, and para-aminobenzoic acid. Some examples of alkylaryl amino acids include ortho-, meta-, and para-aminophenylacetic acid, and γ-phenyl-β-aminobutyric acid.

In one embodiment, the non-naturally occurring amino acids are resistant to common proteases. In one embodiment, the non-naturally occurring amino acids are insensitive to common proteases. Examples of non-naturally occurring amino acids that are resistant or insensitive to proteases include the dextrorotatory (D-) form of any of the above-mentioned naturally occurring L-amino acids, as well as L- and/or D-non-naturally occurring amino acids. The D-amino acids do not normally occur in proteins, although they are found in certain peptide antibiotics that are synthesized by means other than the normal ribosomal protein synthetic machinery of the cell. As used herein, the D-amino acids are considered to be non-naturally occurring amino acids.

In order to minimize protease sensitivity, the peptides may have less than five contiguous L-amino acids recognized by common proteases, irrespective of whether the amino acids are naturally or non-naturally occurring. In another embodiment, less than four continuous L-amino acids are present. In another embodiment, less than three continuous L-amino acids are present. In another embodiment, less than two continuous L-amino acids are present. In one embodiment, the peptide has only D-amino acids, and no L-amino acids.

In one embodiment, the peptide contains protease sensitive sequences of amino acids and at least one terminal amino acid is conjugated to Biotin to reduce this sensitivity. Biotin conjugation to N- or C-ends of the peptide offer steric protection against proteases and thereby, biotin-conjugated peptide confers protease resistance.

Carboxyl groups, especially the terminal carboxyl group of a C-terminal amino acid, may be amidated with, for example, ammonia to form the C-terminal amide. Alternatively, the terminal carboxyl group of the C-terminal amino acid may be amidated with any primary or secondary amine. The primary or secondary amine may, for example, be an alkyl, especially a branched or unbranched C₁-C₄ alkyl, or an aryl amine. Accordingly, the amino acid at the C-terminus of the peptide may be converted to an amido, N-methylamido, N-ethylamido, N,N-dimethylamido, N,N-diethylamido, N-methyl-N-ethylamido, N-phenylamido or N-phenyl-N-ethylamido group.

Cationically-enframed high density aromatic peptides include, but are not limited to, the following peptide examples (where X is any aromatic residue, dC is any cationic residue of a D-amino acid, and mdC is any modified N-terminal NH₂ on alpha-carbon of a cationic residue of D-amino acid):

TABLE 1
mdC-X-X-dC-NH₂-
mdC-X-X-X-dC-NH₂-
mdC-X-X-dC-X-X-dC-NH₂-
mdC-X-X-X-dC-X-X-X-dC-NH₂-
mdC-X-X-X-dC-dC-X-X-X-dC-NH₂-
mdC-X-X-dC-X-X-dC-X-X-dC-NH₂-

Examples of suitable aromatic residues include Phe, Trp, Tyr. Examples of cationic residues include Arg, His and Lys.

TABLE 2
Examples of peptides

dArg-Phe-Phe-dArg (SEQ ID NO: 1, HDAP2)
dArg-Phe-Phe-dLys (SEQ ID NO: 2)
dLys-Phe-Phe-dArg (SEQ ID NO: 3)
dLys-Phe-Phe-dLys (SEQ ID NO: 4)
dArg-Tyr-Tyr-dArg (SEQ ID NO: 5)
dArg-Tyr-Tyr-dLys (SEQ ID NO: 6)
dLys-Tyr-Tyr-dArg (SEQ ID NO: 7)
dLys-Tyr-Tyr-dLys (SEQ ID NO: 8)
dArg-Trp-Trp-dArg (SEQ ID NO: 9)
dArg-Trp-Trp-dLys (SEQ ID NO: 10, HDAP6)
dLys-Trp-Trp-dArg (SEQ ID NO: 11)
dLys-Trp-Trp-dLys (SEQ ID NO: 12)
dArg-Phe-Phe-Phe-dArg (SEQ ID NO: 13, HDAP4)
dArg-Phe-Phe-Phe-dLys (SEQ ID NO: 14)
dLys-Phe-Phe-Phe-dArg (SEQ ID NO: 15)
dLys-Phe-Phe-Phe-dLys (SEQ ID NO: 16)
dArg-Tyr-Tyr-Tyr-dArg (SEQ ID NO: 17)
dArg-Tyr-Tyr-Tyr-dLys (SEQ ID NO: 18)
dLys-Tyr-Tyr-Tyr-dArg (SEQ ID NO: 19)
dLys-Tyr-Tyr-Tyr-dLys (SEQ ID NO: 20)
dArg-Trp-Trp-Trp-dArg (SEQ ID NO: 21)
dArg-Trp-Trp-Trp-dLys (SEQ ID NO: 22)
dLys-Trp-Trp-Trp-dArg (SEQ ID NO: 23)
dLys-Trp-Trp-Trp-dLys (SEQ ID NO: 24)

### EXAMPLES

Biotin-dArg-Phe-Phe-dArg-NH₂ (SEQ ID NO: 1, biotin-HDAP2) penetrates cell membranes.

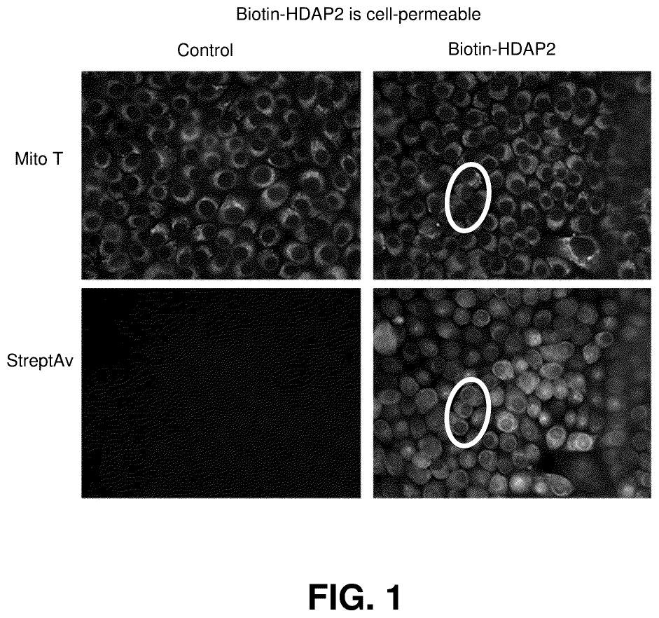

Biotin-HDAP2 was studied using Madin-Darby Bovine Kidney Epithelial cell line (MDBK). Monolayers of cells were grown on 6-well plates for 3 days. On day 4, cells were washed twice with pre-warmed DMEM, and then incubated with 0.2 ml of DMEM containing 10 μM biotin-HDAP2 at 37° C. for up to 1 h. Cells were then washed and fixed in 4% paraformaldehyde and biotin-HDAP2 was detected using Alexa FLUOR™ 488 streptavidin. Cells were also co-stained with MitoTracker-Red or DAPI to identify cytoplasmic compartment or nuclei, respectively. Cells were then visualized in a fluorescent microscope (Nikon Eclipse-ci-SRS). Biotin-HDAP2 was primarily localized to the cytoplasmic compartment of MDBK cells (FIG. 1)

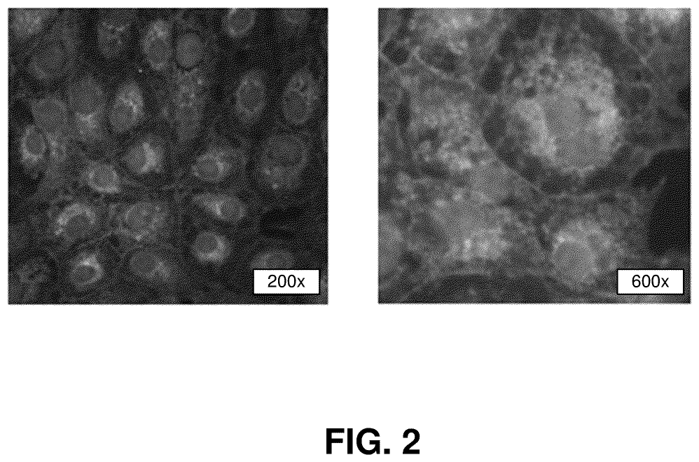

Biotin-HDAP2 is taken into mitochondria (FIG. 2)

Biotin-HDAP2 was studied using Madin-Darby Bovine Kidney Epithelial cell line (MDBK). Monolayers of cells were grown on 6-well plates for 3 days. On day 4, cells were washed twice with pre-warmed DMEM, and then incubated with 0.2 ml of DMEM containing 10 μM biotin-HDAP2 at 37° C. for up to 1 h. Cells were then washed and fixed in 4% paraformaldehyde and biotin-HDAP2 was detected using Alexa FLUOR™ 488 streptavidin. Cells were also co-stained with MitoTracker-Red to identify mitochondria. Cells were then visualized using a fluorescent microscope (Nikon Eclipse-ci-SRS).

Biotin-HDAP2 was co-localized with MitoTracker-Red in MDBK cells.

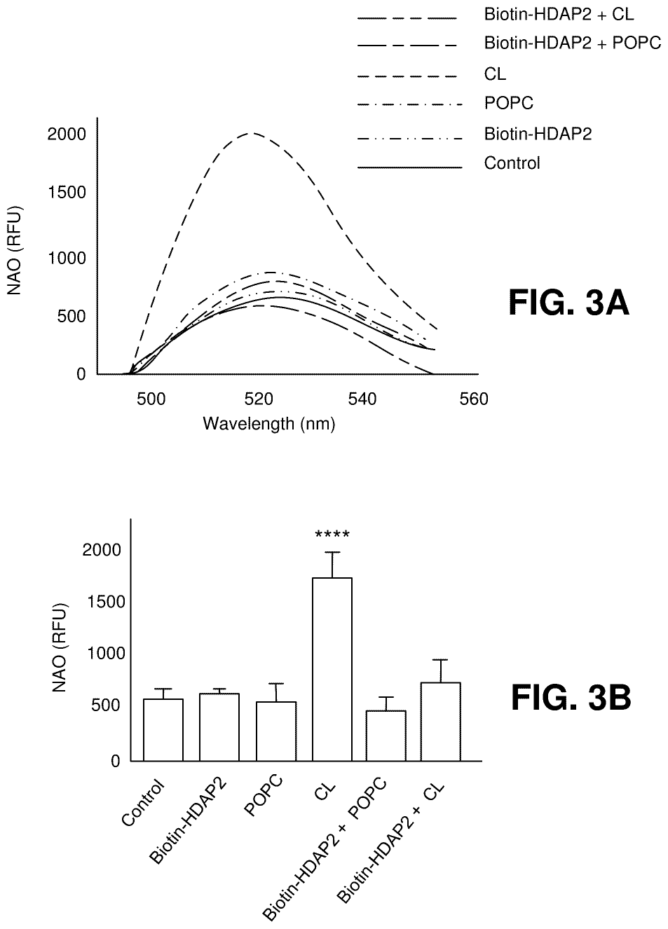

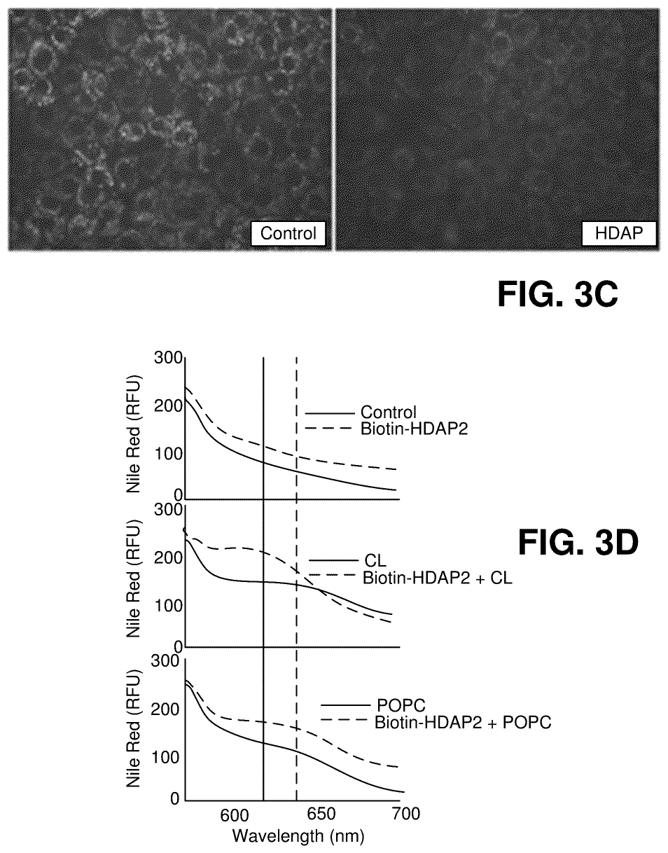

Biotin-HDAP2 was able to inhibit selective cardiolipin marker nonyl acridine orange (NAO) in a dose-dependent manner (FIG. 3A). In addition, biotin-HDAP2 inhibited NAO binding to mitochondria in MDBK cells (FIG. 3B). Finally, interaction of biotin-HDAP2 with cardiolipin produced left shift in a fluorescence on Nile Red, indicating that the peptide binds specifically to the phosphate groups of cardiolipin in a ratio of 1:1 (peptide to cardiolipin) FIG. 3C). This interaction also suggests that biotin-HDAP2₂ bring alkaline environment to cardiolipin, benefiting proton trapping and promoting mitochondrial membrane potential. Furthermore, this interaction allows cardiolipin to become more hydrophobic, which would place it deeper in the inner mitochondrial membrane, promoting negative curvature of mitochondrial cristae and preventing harmful exposure cardiolipin to cytochrome c, destabilization of cytochrome c and mitochondrial electron transport chain. FIG. 3D shows a representative fluorescence emission spectra of Nile Red (1 μM) in the presence of different phospholipids (POPC and CL) alone and with biotin-HDAP2 (i.e. SEQ ID NO: 1 with biotin attached) (ex 540 nm). Right vertical line is at the λₘₐₓ of NR for CL. Left vertical line is at the λₘₐₓ of NR for CL in the presence of biotin-HDAP2. Middle vertical line is at the λₘₐₓ of NR for POPC alone and in the presence of biotin-HDAP2.

Biotin-HDAP2 prevents serum and nutrients starvation induced cell death and degeneration of cellular monolayer.

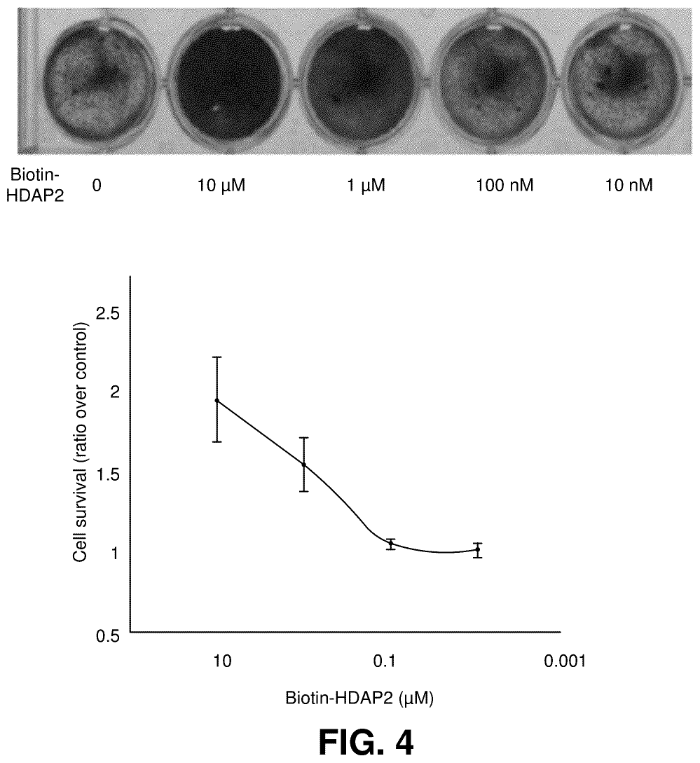

MDBK cells were incubated in serum-free media for 5-7 days in the presence or absence of different concentrations of biotin-HDAP2. At the end of the incubation period, media was removed, the cells were fixed in 4% PFA and then stained with Methylene Blue Loeffler for 30 minutes. Images were obtained using a Tiffen Zoom Camera, interfaced with PC Image, and analyzed using ImageJ NIH software. Experiments were conducted 5 times in triplicates for each experimental condition. Biotin-HDAP2 prevented cell death in a dose-dependent manner with EC50 of about 300 nM (FIG. 4).

Biotin-HDAP2 improves mitochondrial membrane potential in serum- and nutrients-starved cells.

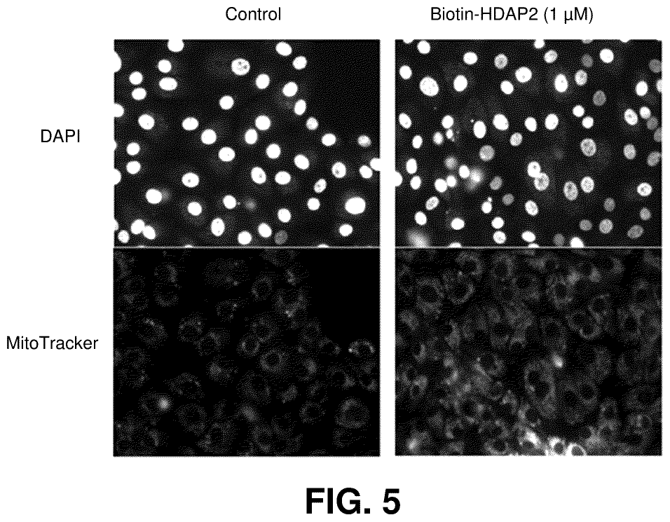

MDBK cells incubated in serum free media for 5-7 days in the presence or absence of 1 μM of biotin-HDAP2. At the end of the incubation period 100 nM MitoTracker was added to the cells and incubated for 15 minutes. Then, media was removed and cells were fixed in 4% PFA. Cells were then visualized using a fluorescent microscope (Nikon Eclipse-ci-SRS). Experiments were conducted 5 times in triplicate for each experimental condition. Biotin-HDAP2 improved mitochondria membrane potential in starved cells (FIG. 5).

Biotin-HDAP2 prevents oxidative stress in serum- and nutrients-starved cells.

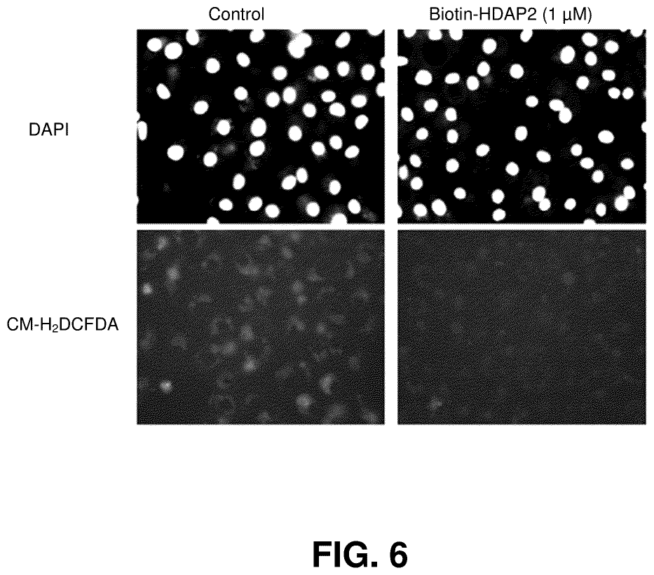

MDBK cells incubated in serum free media for 5-7 days in the presence or absence of 1 μM of biotin-HDAP2. At the end of the incubation period, 100 nM CM-H2DCFDA (oxidative stress marker) was added to the cells and incubated for 30 minutes. Then, media was removed and cells were fixed in 4% PFA. Cells were then visualized using a fluorescent microscope (Nikon Eclipse-ci-SRS). Experiments were conducted 5 times in triplicate for each experimental condition. Biotin-HDAP2 prevented oxidative stress in starved cells (FIG. 6).

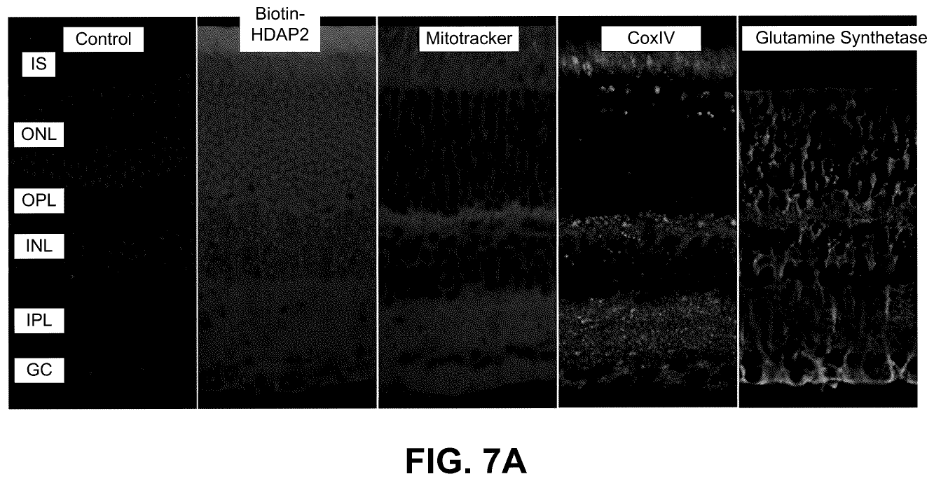

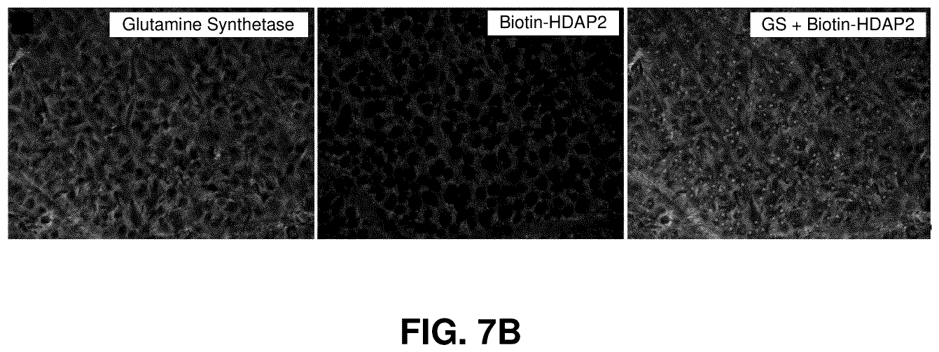

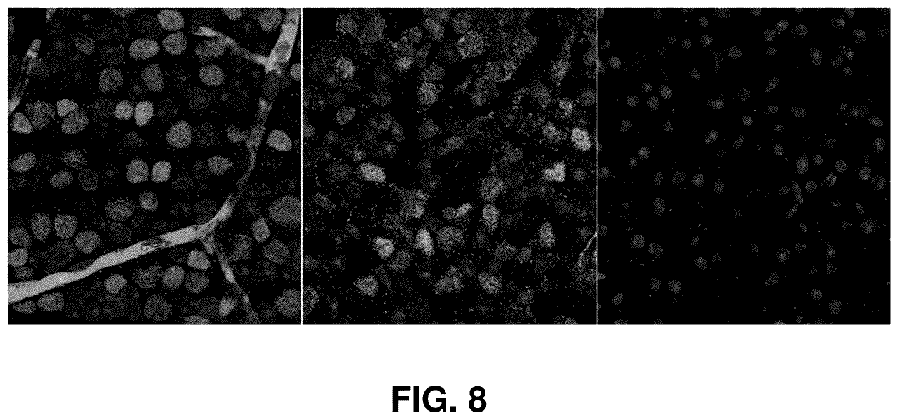

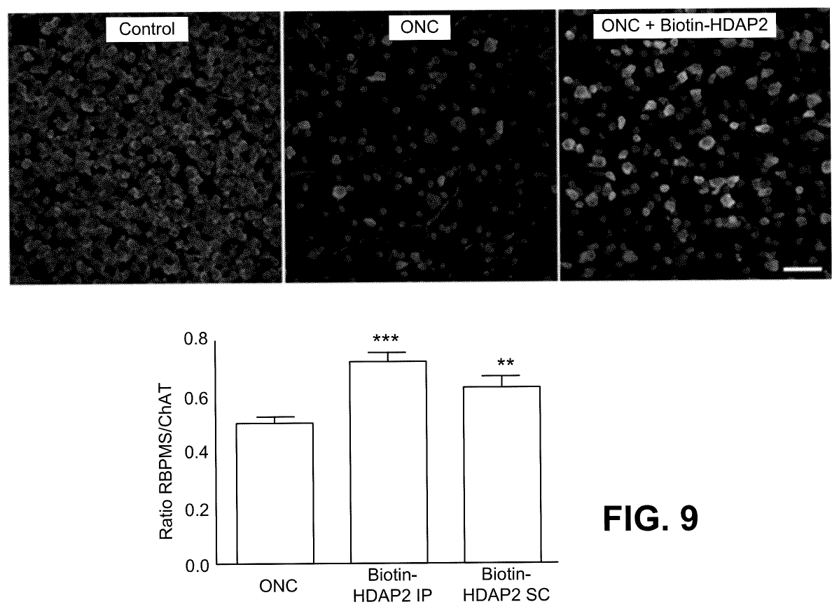

Biotin-HDAP2 (40 mg/kg) was administered intraperitoneally to mice (male or female) and 60 minutes later retina was collected, fixed and sectioned. Biotin-HDAP2 was detected with streptavidin and demonstrated to be widely distributed throughout the retina, including retinal ganglion cells, Müller cells and photoreceptors at the levels far exceeding endogenous biotin label. (FIG. 7A and FIG. 7B). In a second model, biotin-HDAP2 promoted survival of retinal ganglion cells in retinal explants (FIG. 8). FIG. 8 shows explants treated with HDAPs showed more labeled RGCs (green; middle panel) compared to untreated controls (right panel) indicating protection by the peptide. In a third model, biotin-HDAP2 (3 mg/kg) administered either intraperineally, intraocularly or subcutaneously protected retinal ganglion cells following optic nerve crush compared to untreated controls (FIG. 9).

### In Vivo Studies

Once preliminary safety and efficacy of the peptide was established in vitro, biotin-HDAP2 was administered to mice following unilateral optic nerve crush and the distribution and survival of RBPMS-labeled RGCs was assessed. These studies showed that water-soluble biotin-HDAP2 localizes to mitochondria and improves the mitochondrial membrane potential, prevents oxidative stress, and reduces death of cells grown in serum-free culture media. In mice treated systemically with biotinylated peptide, biotin-HDAP2 labeled all layers of the retina in a pattern similar to the mitochondrial marker Cox IV, and that the peptide was colocalized with retinal ganglion cells and Müller glia. Animals treated with biotin-HDAP2 following optic nerve crush showed significantly greater survival of retinal ganglion cells compared to untreated animals at all retinal eccentricities. Biotin-HDAP2 protects cells against serum starvation-induced decreases in mitochondrial membrane potential and leads to significant protection of retinal ganglion cells in the mouse retina following optic nerve crush in vivo. These results suggest that therapeutic agents preserving the mitochondrial membrane potential may help protect neurons following trauma or from neurodegenerative disease.

Biotin-HDAP2 is non-toxic to cells in culture and has no observable adverse effects in mice: Cellular toxicity was assessed in cultured cells by incubating MDBK cells in dilutions of biotin-HDAP2, up 100 μM. No toxicity in MDBK cell growth was observed (data not shown). Toxicity in mice was tested by administering treatment doses (3 mg/kg) daily to animals for 14 days (n>40) or biweekly for two months (n=4) with no adverse effects. None of the animals died, all exhibited normal grooming behaviors and maintained their original weights. Post-mortem analysis of retinal tissue in control eyes showed normal retinal anatomy, without obvious signs of neuronal death, altered mosaics of retinal ganglion cells or disruptions to lamination patterns. Thus, prolonged treatment with biotin-HDAP2 had no detectable toxic effects on the health of mice or damage to the retina.

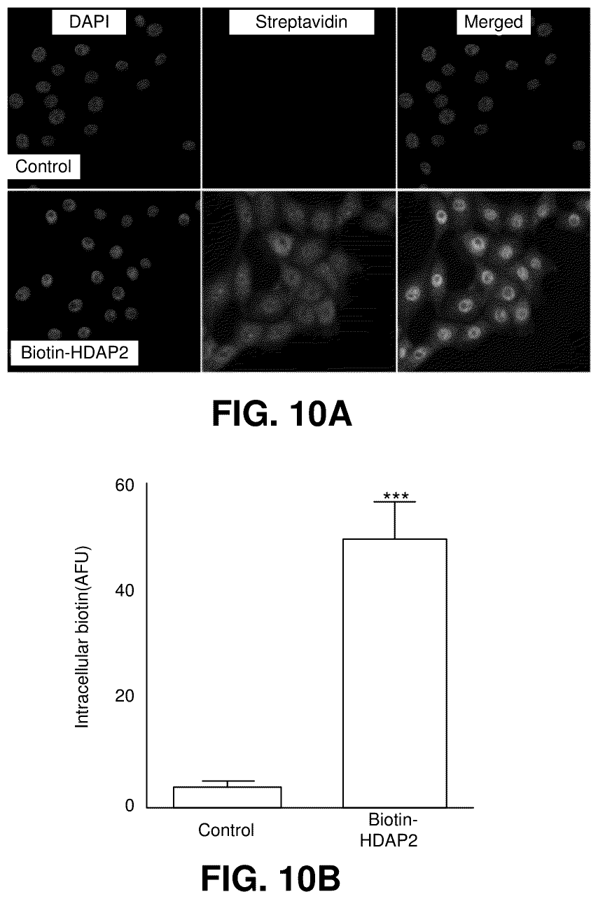

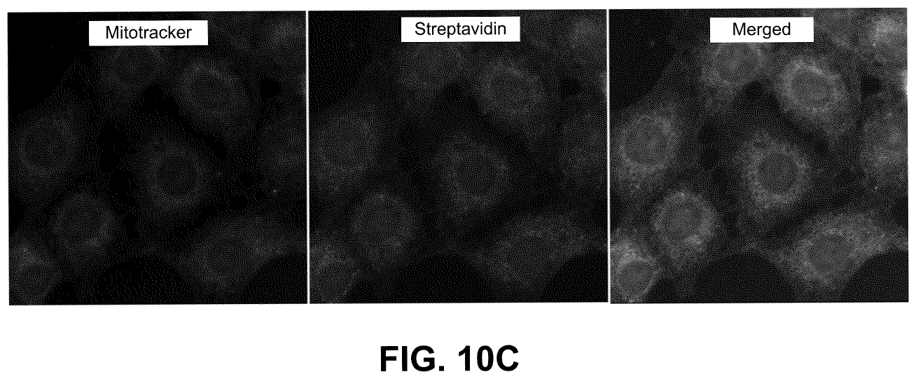

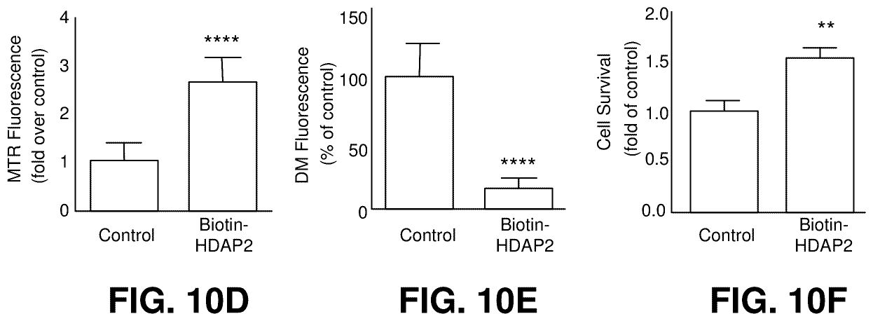

Biotin-HDAP2 co-localizes with mitochondrial markers in cultured cells and improves cell survival following serum starvation: Cultured MDBK cells that were either incubated with biotin-HDAP2 or maintained in control media, were labeled with Hoechst and Alexa Fluor 488 conjugated streptavidin to evaluate the distribution of biotin, either endogenous to the cells or bound to biotin-HDAP2. In both conditions, Hoechst-positive cell nuclei were clearly visible, but only cells incubated with biotin-HDAP2 showed an extensive network of perinuclear streptavidin labeling within the cells, demonstrating extensive uptake (FIG. 10A). Endogenous biotin in the control cells was detectable with streptavidin, but the intensity of fluorescence was significantly weaker than in cells with biotin-HDAP2 (n=4; P<0.001) (FIG. 10B).

When cells were incubated with both biotin-HDAP2 and MTR, a mitochondria potential marker dye that identifies both living and fixed mitochondria, the two markers were colocalized (gray-green color), indicating that biotin-HDAP2 targets mitochondria (FIG. 10C).

Biotin-HDAP2 protects the mitochondrial membrane potential and prevents oxidative stress following serum starvation: Serum starved MDBK cells were used to evaluate the protective effect of biotin-HDAP2 on mitochondria and cell survival. Co-incubation of biotin-HDAP2 (1 μM) with MDBK cells in serum-free conditions for 3-4 days increased the mitochondrial membrane potential in cells by 150% compared to values recorded in control cells in serum-free conditions (P<0.0001) (FIG. 10D). In addition, biotin-HDAP2-treated cells showed significant reductions in CM-H2DCFDA fluorescence, indicating decreased cellular oxidative stress (>80%; P<0.0001) (FIG. 10E) and overall increased cell survival by 50% (P<0.001) (FIG. 10F) compared to untreated controls when MDBK cells were grown in serum-free conditions for 5 days.

Distribution of biotin-HDAP2 peptide in the retina: Systemic administration of biotin-HDAP2 in mice (3 mg/kg, IP) labeled all layers of the retina, at levels exceeding endogenous biotin (FIG. 7A). Since biotin-HDAP2 colocalizes with mitochondrial membranes, retinal sections were labeled with antibodies against the mitochondrial CoxIV for comparison. Biotin-HDAP2 was uniformly distributed throughout the plexiform layers, and matched the pattern of CoxIV labeling, suggesting that biotin-HDAP2 is localized within mitochondria-dense regions of the retina. Vertical and tangential sections through the ganglion cell layer showed heavy biotin-HDAP2 labeling in Müller cell end feet (FIG. 7B) and elevated labeling in RGC somas and axons, where mitochondria are in high density.

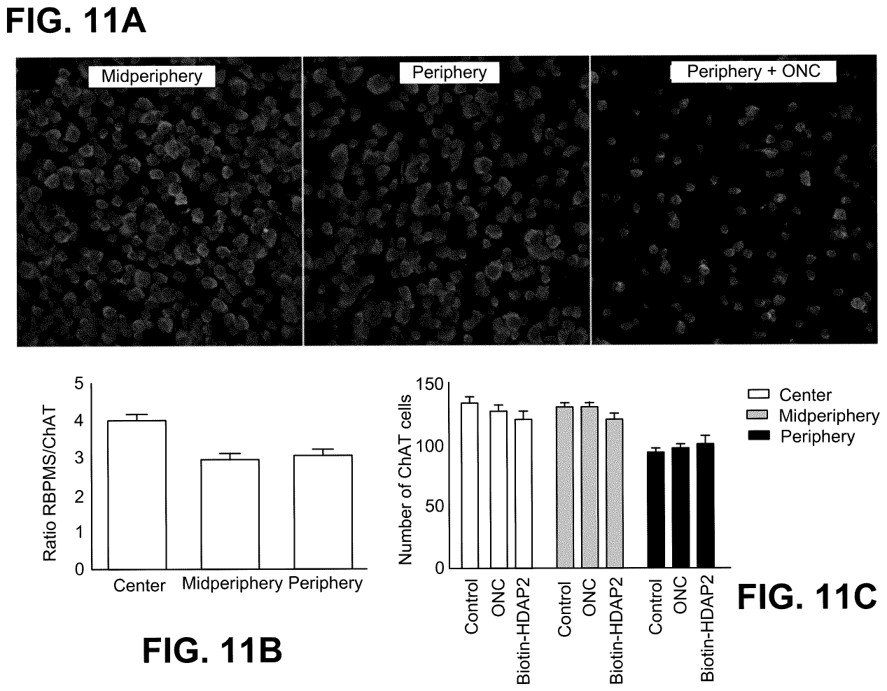

Quantification of Retinal Ganglion Cell Survival: To test whether the protective effects of biotin-HDAP2 in serum-starved cultured cells extended to neurons in vivo, the survival of RGCs following optic nerve crush (ONC) in the presence and absence of the peptide was studied. To control for density differences of RGCs across the retina, values were standardized by comparing ratios of RBPMS-labeled RGCs with the densities of starburst amacrine cells labeled by choline acetyltransferase (ChAT). Starburst amacrine cells, like other displaced amacrine cells, are unaffected by ONC in the short-term. In control tissue, displaced starburst amacrine cells accounted for about 40% of cells in the ganglion cell layer and ratios of RBPMS labeled cells to ChAT neurons was about 4 to 1 in central retina and 3 to 1 at other eccentricities (FIG. 11B). Following ONC, the mosaics of ChAT labeled neurons were not altered by the injury (FIG. 11A) and the density of ChAT cells measured in control, ONC and biotin-HDAP2 conditions were not significantly different from each other (P>0.05, n=11-15 samples per eccentricity) (FIG. 11C), demonstrating their usefulness as an internal control.

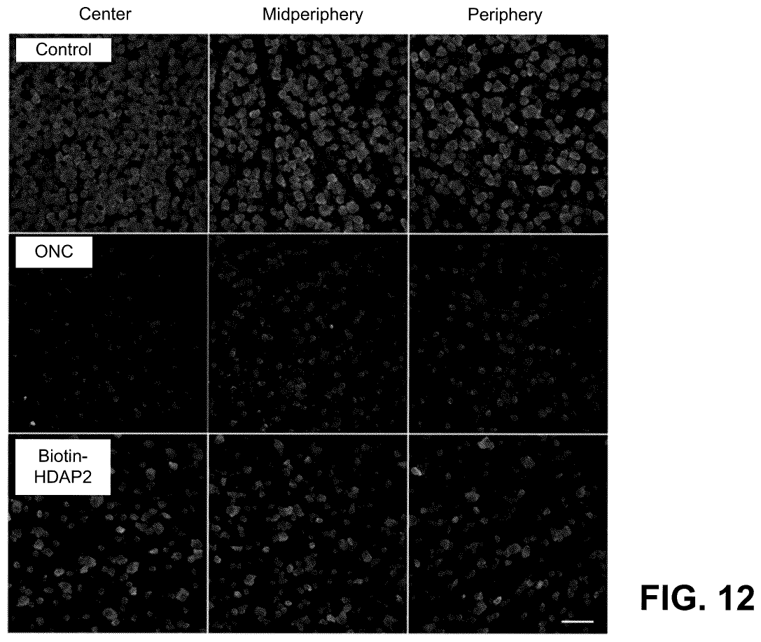

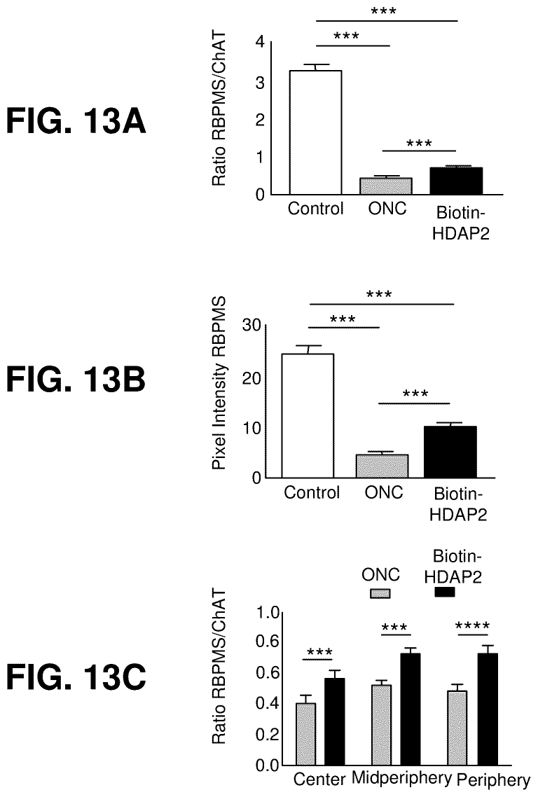

Biotin-HDAP2 protects RGCs following ONC: Crushing the optic nerve resulted in the death of 85% of RGCs within 14 days of the injury (FIG. 12 and FIGS. 13A-C), which is consistent with other studies. In control retinas, the density of RBPMS labeled RGCs decreased predicably from center to periphery (Table 3, 4465±106 cells/mm2 to 2107±126 cells/mm2) (Table 3) and the average ratio of RGCs to starburst amacrine cells was about 3.3 to 1 (FIG. 11B and FIG. 13A). After ONC, the density of RGCs decreased by about 85% (Table 3) and the average ratios of RBPMS to ChAT cells fell to 0.51 to 1 (FIG. 12 and FIG. 13A). RBPMS expression in the tissue also decreased by a similar amount (FIG. 13B). In contrast, ONC animals that were treated daily with biotin-HDAP2 had 40% more RBPMS labeled cells than untreated animals (Table 3), the expression of RBPMS doubled (9.8±0.78 (treated) vs 4.4 +0.45 (untreated); P<0.0001, n=23) (FIG. 13B) and ratios of RGCs to ChAT cells were significantly higher compared to untreated ONC controls (0.70 to 1 vs 0.51 to 1, P<0.001, n=4 for each location) (FIG. 13C).

TABLE 3
Average density of retinal ganglion
cells at varying retinal eccentricity

| | Control | ONC | Biotin-HDAP2 |
|---|---|---|---|
| Central | 4465 ± 106.4 | 408.9 ± 31.21 | 446.1 ± 42.92 |
| Midperipheral | 3070 ± 101.2 | 490.5 ± 36.77 | 694.5 ± 52.61 |
| Peripheral | 2107 ± 126.6 | 391.7 ± 35.87 | 565.4 ± 53.61 |

Treatment with biotin-HDAP2 improved survival of RGCs at all eccentricities studied, but survival improved with increasing distance from the center; in central retina, the percent increase in RBPMS labeled cells was 18%, in midperipheral retina it was 38%, and in the periphery, the numbers of RBPMS-labeled cells was 50% greater than untreated ONC control retinas (FIG. 13C).

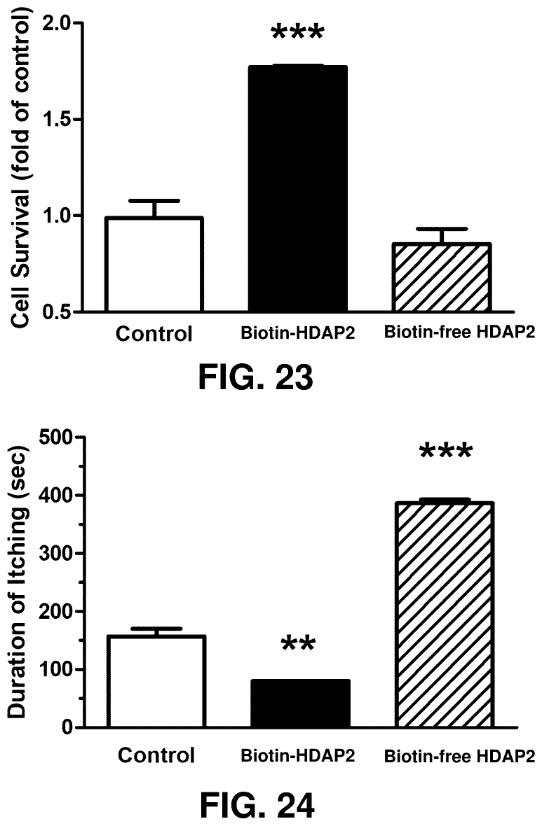

Conclusion: Biotin-HDAP2 is a novel synthetic peptide with a core of Phe-Phe. The Phe-Phe dipeptide promotes formation of electroconductive hydrophobic domains, which optimizes electron transport. However, previous targeting of mitochondria with short peptides containing the Phe-Phe motif resulted in mitochondrial toxicity and cell death. The novel, high density aromatic peptide used in this study brackets Phe-Phe between two Arginine amino acids, to make biotin-HDAP2 both water soluble and non-toxic. The addition of arginine provides overall positive charge to the biotin-HDAP2 analog, which is thought to be important for cell-permeability of peptides and their targeting into mitochondria. Moreover, this new analog was amidated at the C-terminal and had both biotin and D-Arg to protect it against proteolytic degradation. Biotin on the N-terminus of biotin-HDAP2 is not a substrate for biotinidase, so identification of biotin could be used to localize biotin-HDAP2 within the tissue. Biotin-HDAP2 was found to be superior to biotin-HDAP2 for treatment of mitochondria (FIG. 23, FIG. 24).

Biotin-HDAP2 preserved the mitochondrial membrane potential, limited oxidative stress, and improved cell survival during serum starvation in cultured cells: Mitochondria-promoted cellular oxidative stress is commonly mediated by inhibition of the mitochondrial electron transport chain, which results in a subsequent drop in the mitochondrial membrane potential and accumulation of reactive oxidative species (ROS). In the presence of biotin-HDAP2 these effects were not observed. Incubation of MDBK cells with biotin-HDAP2 (1 μM) during serum-starvation significantly improved mitochondrial efficiency and cell viability. In serum-starved cultures incubated with MitoTracker Red CMXRos, a mitochondrion-selective dye that accumulates in active mitochondria, fluorescence intensity increased over 2.5 times when cells were treated with biotin-HDAP2 compared to untreated controls, demonstrating enhanced mitochondrial membrane potential. The improved potential was accompanied by decreases in oxidative stress measured with the general oxidative stress indicator, CM-H2DCFDA, which showed >80% reduction in CM-H2DCFDA fluorescence. Confirmation of biotin-HDAP2's beneficial action was improved cell survival, which increased by 50% in serum-deprived conditions compared to untreated control cells.

Survival of retinal ganglion cells: Another important finding was that mitochondrial-directed biotin-HDAP2 significantly improved the survival of retinal ganglion cells, at all eccentricities in the retina, following optic nerve crush. The biotinylated peptide was identified in the retina after both intravitreal injection and via systemic administration, demonstrating that water soluble biotin-HDAP2 can penetrate the retina-blood barrier and reach mitochondria in retina neurons and glia. Since biotin-HDAP2 localizes with known mitochondrial markers, it is likely that the peptide is targeting mitochondria inside animal cells, as it does in cultured cells, to help to improve mitochondrial function. RGCs are among the most metabolically demanding cells in the central nervous system, and they contain correspondingly high concentrations of mitochondria, particularly in unmyelinated intraretinal axons. Following physical damage to the optic nerve, energy demands on RGCs increase as they attempt to repair axonal damage and reestablish cellular homeostasis. However, as ATP production by mitochondria increases, ROS generation accelerates, which leads to catastrophic cellular effects, including impaired ATP production, mutated mitochondrial DNA, dysregulation of intracellular Ca²⁺, and activation of apoptotic pathways. Mitochondria can function normally in the short-term, but over time, cellular homeostasis is impaired by excessive ROS and leads to mitochondrial dysfunction and death.

Increases in RBPMS expression correlates with improved RGC survival: When the intensity of the confocal laser was controlled, the average expression of RBPMS in the biotin-HDAP2 treated retinas was double compared to the expression of the label in untreated retinas following ONC. RBPMS selectively labels RGCs in many species and its expression decreases in cells following hypoxia, increased intraocular pressure, and after optic nerve crush. These decreases have been correlated with increased expression of caspase 3, a protein that initiates a major apoptotic pathway. When RGC axons are injured, cellular mitochondria experience a drop in the mitochondrial potential, which results in translocation of the pro-apoptotic gene Bax, induction of mitochondrial permeabilization, and activation of caspase-dependent apoptotic programs. BAX can be inhibited by BclX_{L} an anti-apoptotic member of the Bcl2 gene family that prevents serum starvation-induced decreases in the mitochondrial membrane potential in cells. When BCLX_{L} was delivered to animals, it protected the optic nerve and improved RGC survival in DBA/2J mice, who otherwise experienced massive degeneration of RGCs with advancing age. The gene conferred greater protection than genetic deletion of Bax alone, indicating that preservation of the mitochondrial membrane potential may be a better strategy for protecting RGCs following trauma or disease, than direct prevention of apoptosis.

Impact of retinal eccentricity on RGC survival: Biotin-HDAP2 improved survival of RGCs at all retinal eccentricities compared to untreated ONC controls but survival was greatest in areas outside central retina (FIG. 13B); survival was only 18% greater in central retina compared to 50% greater in peripheral retina (P<0.0001). This result is consistent with earlier studies demonstrating that more RGCs survive optic nerve injury when the damage is furthest from the optic nerve head. A high fraction of surviving cells had large somas, which suggests they could be alpha RGCs (FIG. 12). This observation is consistent with recent studies showing that ON-sustained and ON-transient alpha-ganglion cells are more likely to survive optic nerve crush injury compared to other ganglion cell types. Interestingly, ON-alpha cells in mouse retina have an uneven distribution with highest densities in peripheral temporal retina, which are the same areas were most protected by the biotin-HDAP2 peptide.

The subtype-specific survival of RGCs following ONC may be due to differences in gene activation, variations in the concentration of mitochondria, or retinal eccentricity, but survival could also be dependent on the relative health of Müller cells which also depend heavily on the actions of mitochondria. Müller cells provide essential support to RGCs by delivering nutrients and growth factors, and removing metabolic wastes also participate in retinal homeostasis by balancing extracellular ion concentrations and neurotransmitters, such as glutamate. Unlike RGCs, which are most densely concentrated in central retina, Müller cells are homogenously distributed without regard to eccentricity. With a higher ratio of retinal ganglion cells to Müller cells in central retina, elevated oxidative stress would impact Müller cells in central retina more significantly than those in the periphery, where the workload would be more manageable. biotin-HDAP2 labeling was observed in Müller cells (FIG. 7A and FIG. 7B) so it is conceivable that improved mitochondrial function as well as higher concentrations of Müller cells in peripheral retina improved RGC survival.

### Cardiolipin Interaction Studies

Combining Phe-Arg and Phe-Phe motifs in biotin-HDAP2 promotes an interaction of biotin-HDAP2 with cardiolipin. As it was previously demonstrated for the cardiolipin-binding peptide SS-31, biotin-HDAP2 outcompeted an interaction of the selective cardiolipin probe, NAO, with CL in a dose-dependent manner. Biotin-HDAP2 binds to CL in a 1:2 ratio, which indicates stronger and more specific binding than found for SS peptides and CL, which have a 1:1 ratio. Phe is thought to penetrate deeply into the cardiolipin-containing phospholipid bilayer, which would increase hydrophobicity of acyl groups in the phospholipid bilayer. Biotin-HDAP2 was found to selectively promote a NR blue shift in CL-containing POPC liposomes, without affecting POPC liposomes on their own.

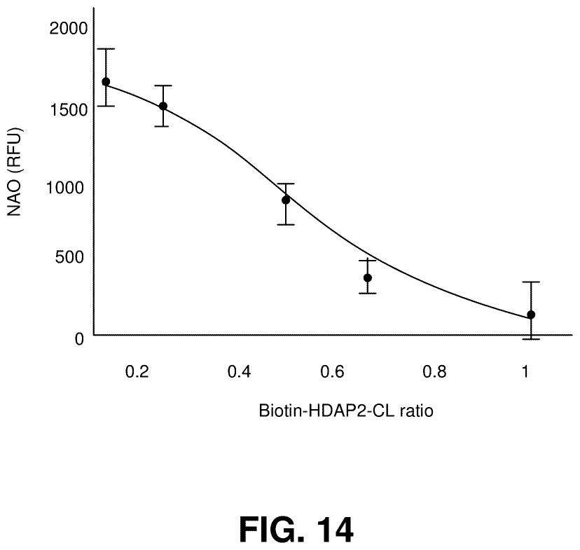

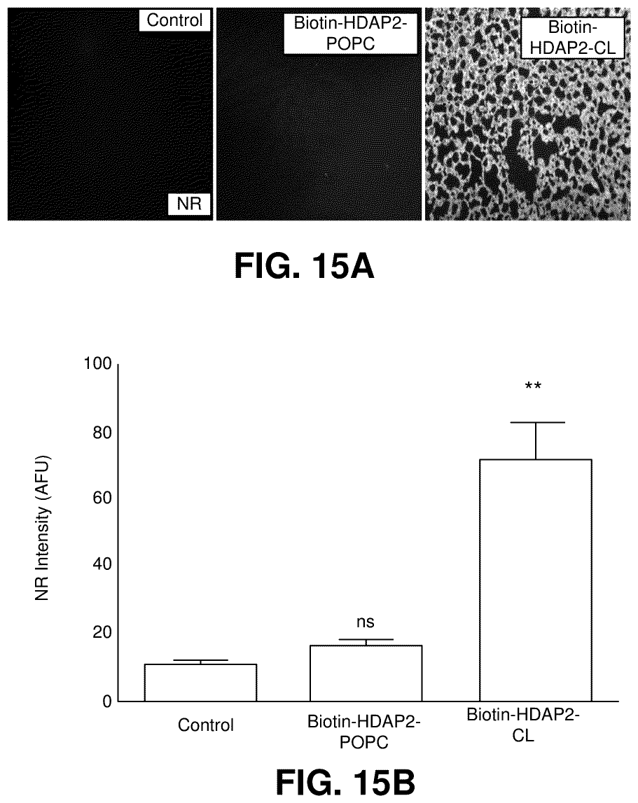

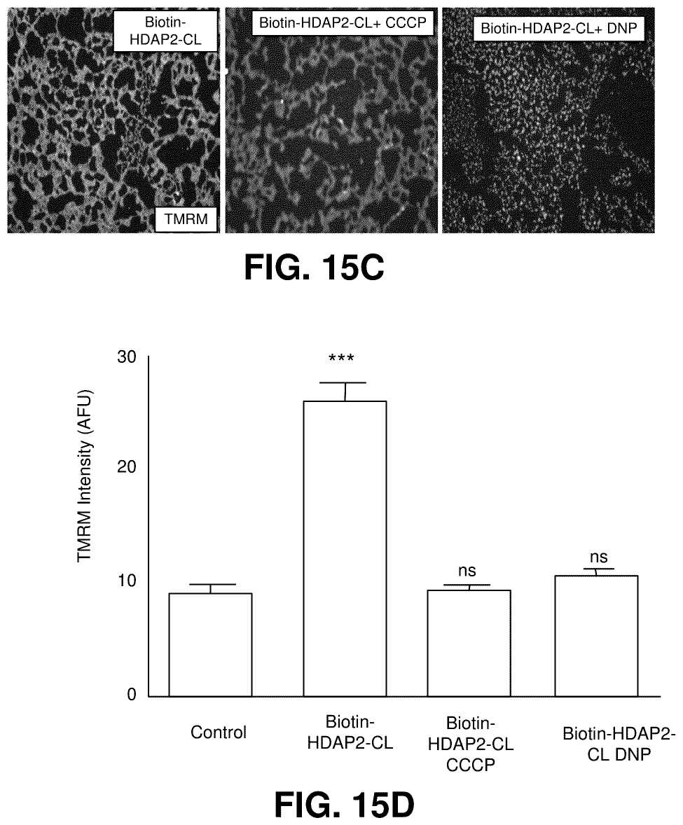

The selectivity of biotin-HDAP2 for CL was demonstrated through the displacement of a known CL-selective fluoroprobe, nonyl acridine orange (NAO), by biotin-HDAP2 from POPC-CL liposomes (FIGS. 15A-C). When NAO was mixed with CL, the intensity of its fluorescence increased by about 3-fold, compared to the intensity of fluorescence when NAO was mixed with POPC or biotin-HDAP2 alone (FIG. 3A, FIG. 3B and FIG. 14). When also combined with CL, biotin-HDAP2 decreased the intensity of NAO fluorescence to background levels, and did not interfere with the interaction between NAO and the POPC liposomes (FIG. 3A and FIG. 3B), suggesting the selectivity of biotin-HDAP2 to CL. In addition, biotin-HDAP2 outcompeted NAO for binding with CL, with EC50 at a ratio of about one peptide to two CL molecules, suggesting that biotin-HDAP2 can bind two CL simultaneously. The selective interaction of biotin-HDAP2 with CL was confirmed by using the dye Nile Red (NR), which selectively binds to acyl chains of phospholipids by producing a blue shift in its emission. In POPC-CL liposomes, biotin-HDAP2 promoted a blue shift of NR from 638 nm to 618 nm (FIG. 3D middle panel), but no shift could be detected when biotin-HDAP2 was added to POPC liposomes (FIG. 3D, bottom panel), confirming the selectivity of biotin-HDAP2 for CL.

The blue shift observed from the NR experiments suggests that biotin-HDAP2 increases the hydrophobicity of the acyl chain in CL and would also indicate that d-Arg is likely to be at the CL head in the lipid-water interphase. Arg would provide positive charge to a biotin-HDAP2-CL complex, which can be bound to negatively charged surfaces. To test this hypothesis, CL and biotin-HDAP2 were combined without additional POPC. Surprisingly, biotin-HDAP2-CL vesicular structures adhered to the negative surface of the cell culture wells and were stained with NR, unlike blank wells that were unstained (FIG. 15A right panel and FIG. 15B). Vesicular structures or NR staining were not observed when biotin-HDAP2 was mixed with POPC in the blank wells (FIG. 15A and FIG. 15B) or when added to untreated wells (data not shown), further demonstrating selectivity of biotin-HDAP2 for CL.

Concentration of alkaline Arg on the surface of biotin-HDAP2-CL vesicles could potentially trap protons on CL to create polarization of a biotin-HDAP2-CL membrane. TMRM, a proton-gradient transmembrane potential probe commonly used in mitochondria research, was used to show that biotin-HDAP2-CL vesicles were stained (FIG. 15C left panel and FIG. 15D), indicating a transmembrane proton potential across the membrane of the vesicles. To confirm that the potential across the membrane was due to proton trapping of the CL-biotin-HDAP2 surface, significant decreases in TMRM staining in the presence of proton ionophores, carbonyl cyanide m-chlorophenylhydrazone (CCCP) and dinitrophenol (DNP) were measured (FIG. 15C (middle and right panels and FIG. 15D). This data supports the idea that biotin-HDAP2 traps protons on biotin-HDAP2-CL complex and generates a transmembrane proton potential.

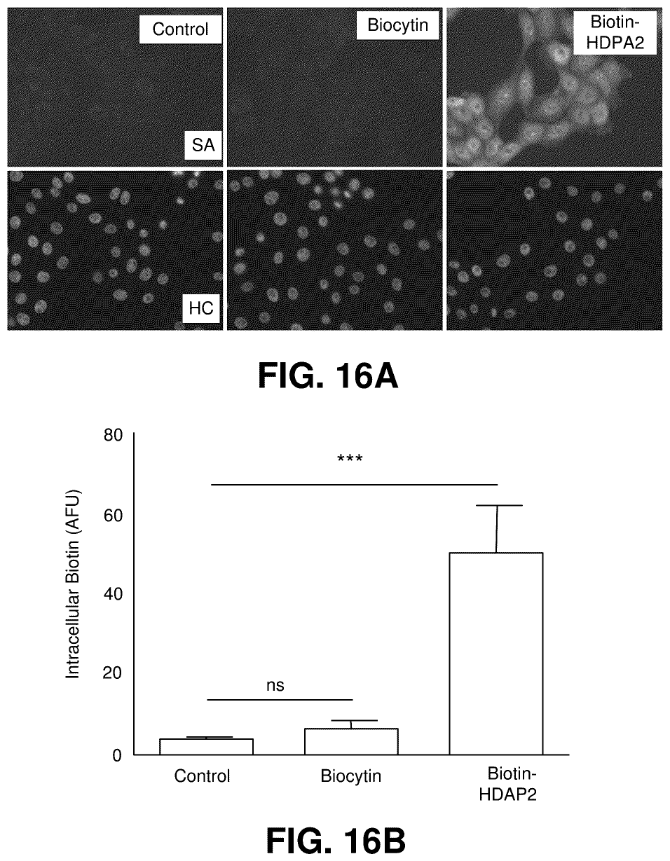

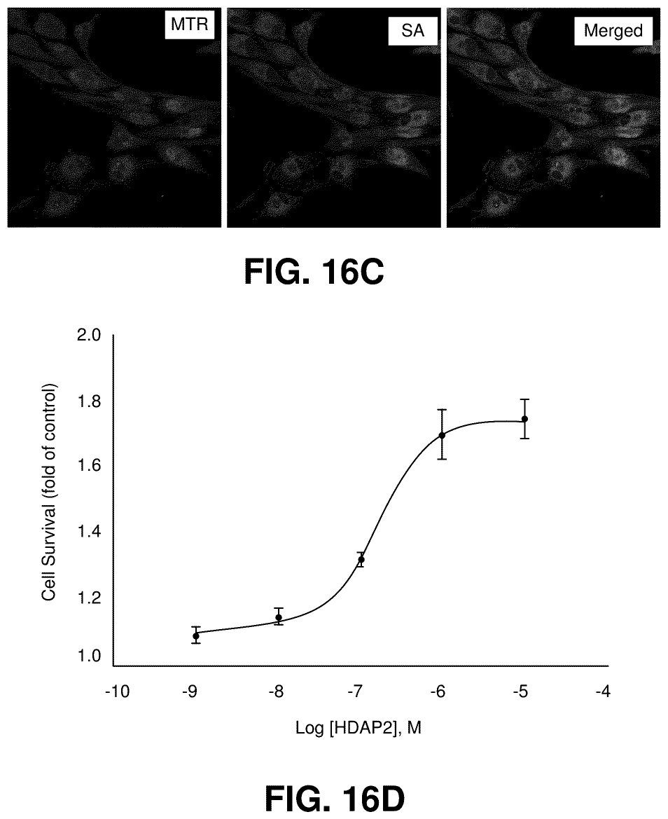

Biotin-HDAP2 could be taken up into MDBK cells. Cells dramatically deplete mitochondrial biotin after 3 days of incubation in serum-free DMEM, yet biotin-HDAP2 was taken up into the serum-starved cells after just 1 hour of exposure with the peptide (FIG. 16A right panel and FIG. 16B). Biotin-HDAP2 was distributed in the perinuclear area, as indicated by streptavidin staining around nuclei (stained by Hoechst 33342) and entered the cell about 6 times faster than observed for biocytin (FIG. 16A middle panel and FIG. 16B), suggesting that mechanisms used to take up biotin-HDAP2 into cells are likely to be peptide structure dependent. To determine mitochondria-targeting of biotin-HDAP2, biotin-HDAP2 is colocalized with the selective mitochondrial marker, MitoTracker red CMXRos (FIG. 16C) and promoted cell survival of MDBK cells in serum-free conditions with EC50 of about 100 nM (FIG. 16D).

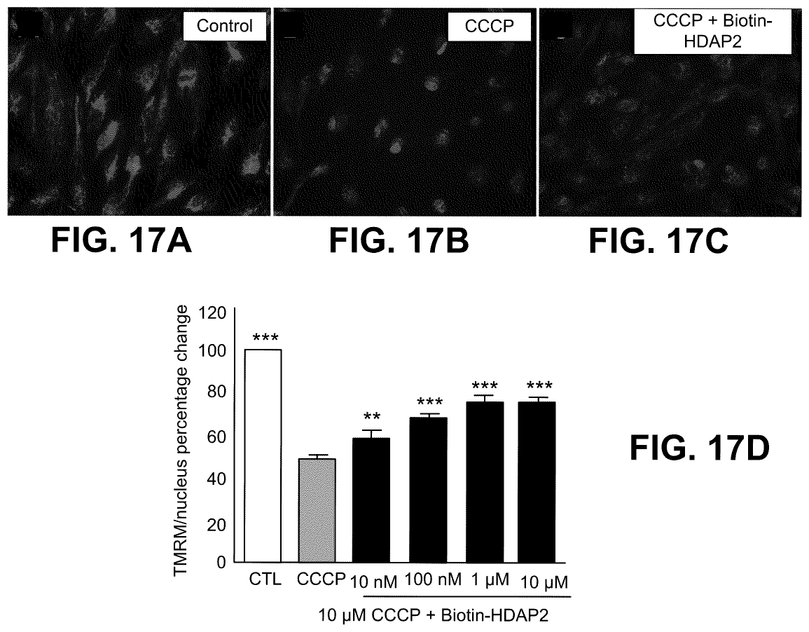

Optimization of proton trapping within the biotin-HDAP2-CL complex on the inner mitochondrial membrane (IMM) may have a potential to provide some resistance to the mitochondrial uncouplers. To test this hypothesis, the effect of biotin-HDAP2 on the ΔΨm decrease in the presence of known mitochondrial uncoupler CCCP in ARPE-19 cells was examined. These cells were specifically chosen for their robust mitochondria and their ability to survive even when exposed to mitochondrial stressors. This allowed rapid changes in ΔΨm to be studied without inducing cell toxicity from overlapping events. Addition of even 10 μM of CCCP dropped the ΔΨm to 20% of control cells (FIG. 17A and FIG. 17B). Biotin-HDAP2 prevented mitochondrial depolarization in a dose-dependent manner when incubated together with 10 μM of CCCP (FIG. 17A and FIG. 17B), suggesting that biotin-HDAP2-mediated proton trapping might be a novel recoupling mechanism to preserve the ΔΨm.

### Materials and Methods for In Vitro Studies

Cells and media: Tissue culture studies were carried out using Madin-Darby Bovine Kidney (MDBK) cells (Lot ATCC-CCL22) derived from primary tissue. MDBK cells were plated and grown in Dulbecco's Modified Eagle's Minimum Essential Medium (DMEM, Cytiva) with 4.5 g of glucose and 10% horse serum, and held in an incubator with 5% CO₂ at 37° C.

Biotin-HDAP2 uptake: To assess uptake of biotin-HDAP2 into cells, cultures were incubated in serum-free media for three days to deplete endogenous biotin (Birk et al., 2013) and then treated with biotin-HDAP2 (1 μM) for one hour. Cells were fixed with 4% paraformaldehyde and incubated for one hour with Alexa Fluor 594 conjugated streptavidin (1:100, Jackson ImmunoResearch) to localize the peptide within the cells, Hoechst 33342 (10 μg/ml, Novus) to label nuclei, and Mito Tracker Red CMXRos (MTR; 50 nM, Invitrogen) to identify mitochondria; MTR binds to thiol groups, which are heavily concentrated in the mitochondrial matrix. Cells were rinsed and imaged immediately in the dish using a Nikon Eclipse 50i fluorescent microscope with a 40× immersion objective (NA 0.8).

Serum starvation experiments: Biotin-HDAP2's ability to improve cell viability and function following serum starvation was assessed. For all experiments, MDBK cells were seeded in 35 mm cell culture dishes (5×10⁴ cells) in DMEM medium with 10% FBS (Fetal Bovine Serum) for 24 hrs and then incubated in serum-free medium with or without biotin-HDAP2 (1 μM) for 5 days to induce cell death. For quantitative measurements, wells were washed to remove dead cells, and then stained with AlamarBlue for 60 min. Quantitative analysis of dye conversion was measured using a fluorescent plate reader (550 ex/580 em) and cell viability was expressed as folds change (increase or decrease) over scrum-starved untreated cells.

To measure the effects of biotin-HDAP2 on the mitochondrial potential and cellular oxidative stress, cells were cultured in DMEM/F12 without fetal bovine serum (FSB) in the presence or absence of biotin-HDAP2 (1 μM). After 3 days, nuclei were labeled with Hoechst 33342 (10 μg/ml) and cells were incubated for 15 minutes at 37° C. with MTR (100 nM) to evaluate the strength of the mitochondrial potential or were incubated with the general oxidative stress indicator CM-H2DCFDA (5 μM, Invitrogen, Carlsbad, CA) for 15 min at 37° C. In each instance, cells were imaged immediately after staining and prior to fixation.

Unless otherwise noted, data for all tissue culture experiments were collected from three independent trials, with each experiment done in triplicate. For each experiment, 4-6 fields of cells were randomly selected and imaged using a Nikon Eclipse 50i fluorescent microscope with a 40X immersion objective (NA 0.8) and using filters specific for either green or red wavelengths as appropriate (ex488 nm/ex515-540 nm). Photooxidation of the indicator dyes were minimized by using single exposures to the fluorescence for each field imaged. Fluorescence intensity was analyzed using ImageJ (NIH), and MTR and CM-H2DCFDA fluorescence were normalized to Hoechst fluorescence to account for differences in cell number in each field. Differences among groups were compared by Student's t-test and P values less than 0.05 were considered significant. All data are expressed as means±SEM.

Animal Experiments: Adult C57BL/6 mice (The Jackson Laboratory, Bar Harbor, ME) of either sex, 2-6 mos. of age were studied. These were bred and housed in the animal facility at York College, CUNY. All procedures using animals were approved by the York College IACUC and were consistent with the ARVO Statement for the Use of Animals in Ophthalmic and Vision Research.

Biotin-HDAP2 uptake in retina: Adult animals were injected with 50 mg/kg biotin-HDAP2 (IP; GenScript, Piscataway, NJ) reconstituted in saline and observed for 2 hours before sacrifice. Eyes were marked to record their orientation, and the animals were given an overdose of ketamine (300 mg/kg)/xylazine (60 mg/kg) (IP) before removing each eye from the orbit with forceps. Whole eyes were dropped into 4% paraformaldehyde that was constituted in either 0.1M Tris or phosphate buffers (pH 7.3) and after ten minutes, the cornea, lens and vitreous body were removed to ensure better penetration of the fixative into the retina. The eyecups were returned to the fixative for one hour. Retinas for wholemounts were dissected from the pigment epithelium by cutting along the or a serrata, gently teasing the retina away from the back of the eye and then freeing the retina by severing the optic nerve. Retinas for cryo-sectioning remained in the eyecup, were cryoprotected in 0.1M Tris or phosphate-buffered sucrose (10%, 20% and 30%) prior to embedding in OCT (Polyfreeze, Polysciences) and sectioned at a thickness of 14 μm at −25° C. using a cryostat (Leica CM3050S). Uptake of biotin-HDAP2 was revealed by incubating the tissue (wholemounts and sections) with Alexa Fluor-conjugated streptavidin (1:200, Jackson ImmunoResearch) either overnight (sections) or for three days (wholemounts) and counterstained with various antibodies (described below).

Optic Nerve Crush Procedure: Adult mice (2-6 mo.) were anesthetized with a cocktail of ketamine (85 mg/kg) and xylazine (20 mg/kg) (IP), the corneas were numbed with a drop of 0.5% proparacaine hydrochloride ophthalmic solution (Falcon Pharmaceuticals), and the animals were positioned under a Nikon dissecting microscope, dorsolaterally either on the left or right sides. The dorsomedial conjunctiva was cut with microdissection spring scissors, the intraocular muscles were separated, and the eyeball was slightly retracted to expose the optic nerve. Fine, self-closing forceps (Roboz, RS-5027) were used to clamp the optic nerve 1-2 mm behind the globe, for approximately 3 seconds without applying additional tension. The eye was repositioned within the orbit, the conjunctiva reflected over the extraocular eye muscles and the incision covered with ophthalmic polymyxin B-neomycin-bacitracin ointment (3.5 mg/gm, Bausch and Lomb). The optic nerves from the contralateral eye were untouched and used as untreated controls. Following the crush procedure, mice were given a subcutaneous injection of extended-release buprenorphine (0.05 mg/kg, Ethiqua XR) for analgesia and placed on a heating pad until awake and grooming, at which point they were returned to their home cages. Half the animals were given biotin-HDAP2 immediately following the procedure and then daily thereafter (3 mg/kg, IP) for 14 days.

Immunohistochemistry: Fixed retinas were labeled with cell markers using conventional immunohistochemical protocols to identify cells and structures associated with biotin-HDAP2, and to identify retinal ganglion cells and starburst amacrine cells to assess cell survival following optic nerve crush. Retinas and sections were blocked and permeabilized with 4% normal donkey serum in 0.1M Tris or phosphate buffers with 0.5% Triton-X for 1 hour and then incubated overnight (for sections) or for 3 days (for wholemounts) at 4° C. in selected primary antibodies, and 2% normal donkey serum in the same buffer. The tissue was rinsed 3×10 minutes with buffer and then incubated with secondary antibodies for one hour at room temperature (sections) or overnight at 4° C. for wholemounts. Antibodies and markers used were rabbit anti-RBPMS (1:200; Invitrogen, PA5-31231) to label all retinal ganglion cells; goat anti-choline acetyltransferase (ChAT 1:100; EMD Millipore, AB144P) to label starburst amacrine cells; rabbit anti-glutamine synthetase (1:200; Invitrogen, PA1-46165) to label Müller cells; and rabbit anti-cox4 (1:200; Invitrogen, PA5-29992) to identify mitochondria. Secondary antibodies were raised in donkey and conjugated with either Alexa Fluor 488, 594 or 647 (1:500, Life Sciences). To identify the distribution of cell nuclei, and to delineate retinal layers, tissue was labeled with the nuclear stain ToPro3 (1:1000; Invitrogen) for 15 minutes. After staining, sections and retinas were rinsed in buffer and coverslipped in Vectashield mounting media (Vector Labs) to prevent photobleaching.

Imaging and Data Analysis: Retinal cells were imaged using an Olympus Fluoview 300 confocal microscope with an Olympus 40× oil immersion objective (NA 1.0) in dorsal, ventral, nasal and temporal regions of the retina at each of three eccentricities approximately 1, 2 and 3 mm from the optic disk. The laser intensity for the green channel was set at ˜530V so that RBPMS expression could be compared between treatment conditions. In central regions, Z-stacks (1 μm steps) were collected through the ganglion cell layer to ensure that RGCs above and below the middle focal plane were imaged and a through focus projection was generated and used to count cells. In midperipheral and peripheral areas of the retina, single images were collected instead because cells in the ganglion cell layer were visible in a single focal plane. Each site was imaged separately using each of three wavelengths (488, 594, and 647 nm) and then merged in Photoshop to observe RGCs, starburst cells and cell nuclei in the same fields. Labeled cells in each image field (350 μm×350 μm) were counted manually using ImageJ (NIH) while blinded to the experimental condition and used to compute the densities of RGCs and the ratios of RBPMS to ChAT cells for each location and condition. RBPMS expression in the tissue was quantified using the “Analyze” feature inImageJ to compute the mean pixel intensity (in arbitrary fluorescence units) from the confocal images.

### Materials and Methods for Cardiolipin Interaction Studies

Chemicals: Biotin-HDAP2 (>96.5% pure) was synthesized by GeneScript (Piscataway, NJ). Other chemicals, including 1-palmitoyl-2-oleoyl-sn-glycero-3-phosphocholine (POPC), cardiolipin from bovine heart (CL, comprising primarily of tetralinoleoyl CL), reagents and assay kits were purchased from Sigma Aldrich (St. Louis, MO).

Preparation of Liposomes: Lipids in chloroform were combined in 12×75 mm glass tubes with either 150 μM CL: 150 μM POPC or 300 μM POPC. The solvent was allowed to slowly evaporate, which resulted in a lipid film that could be rehydrated in an aqueous solution of 10 mM HEPES, pH 7.4. The resulting multilamellar vesicles were vortexed lightly and sized into small unilamellar vesicles by heated bath sonication for 25 min. All liposomes were cooled to ambient temperature before use.

Interaction of biotin-HDAP2 Peptide with Phospholipids: The ability of biotin-HDAP2 to displace nonyl acridine orange (NAO), a fluoroprobe known to selectively bind CL was determined. Interaction of biotin-HDAP2 with phospholipids was examined by measuring changes in the fluorescence spectra of 3 μM NAO (Molecular Devices, Sunnyvale, CA)) with excitation at 480 nm. CL and POPC in liposomes were use at 30 μM. Dose response curves were done with 30 μM CL, biotin-HDAP2 (0, 2.5, 5, 10, 20, and 30 μM), and 3 μM of NAO (ex/em at 480 nm/520 nm). All experiments were done in 10 mM Hepes (pH 7.4) to optimize electrostatic interactions of peptides with phospholipids. The different solvents used to dissolve the various phospholipids (chloroform, methanol, and ethanol) had a negligible effect on the fluorescence spectra.

Interactions between biotin-HDAP2 and Phospholipid liposomes measured by Nile Red fluorescence: The hydrophobicity of biotin-HDAP2 and phospholipids was examined by changes in the spectral fluorescence shift and intensity of Nile Red (NR, 10 μM). NR increases its intensity of fluorescence when combined with the hydrophobic environments of acyl chains of phospholipids, and produces a blue shift when hydrophobicity is increased. All experiments were done with 30 μM of either CL or POPC and 30 μM of biotin-HDAP2 in 10 mM Hepes (pH 7.4) to optimize the electrostatic interactions of peptides with phospholipids. The solvents used to dissolve the various phospholipids (chloroform, methanol, and ethanol) had negligible effects on the fluorescence spectra.

Preparation and Visualizing biotin-HDAP2-CL vesicles with Nile Red: To visualize biotin-HDAP2-CL vesicles with NR, 30 μM of CL or POPC were mixed with 100 μM of biotin-HDAP2 in 10 mM Hepes pH 7.4. The mixture was sonicated for 30 min, transferred to 35 mm cell culture wells, and allowed to settle for 24 hours. The next day, 10 μM of NR was added for 15 min at room temperature and then gently washed three times to remove excess NR. Samples with no peptide or phospholipids were used as controls. Fluorescence staining was observed by using a Nikon Eclipse 50i. The intensity of NR fluorescence was measured in each field, using the “Analyze” feature in ImageJ to compute the mean pixel intensity (in arbitrary fluorescence units) from the images. Differences among groups were compared by one-way ANOVA. Post hoc analyses were carried out using Tukey's multiple comparisons test.

Labeling biotin-HDAP2-CL vesicles with TMRM: After forming biotin-HDAP2-phospholipids vesicles, 100 nM of TMRM was added for 15 min at room temperature and then gently washed three times to remove excess TMRM. The sample with no peptide or CL was used as control. For testing proton-gradient uncouplers, 100 μM CCCP or 1 mM DNP was added together with TMRM and imaged and analyzed as described for NR.

Intracellular Localization of biotin-HDAP2: Intracellular targeting of biotin-HDAP2 was determined by incubating MDBK cells (ATCC-CCL22) that were grown in biotin-free media for 72 hours, with 10 μM of N-biotinylated biotin-HDAP2 for 1 hour. Cells were then fixed with 4% paraformaldehyde, permeabilized with 0.1% Triton X-100, and treated with streptavidin conjugated with Alexa Fluor 488 (SA) for 30 minutes at room temperature. The Hoechst 33342 probe was used to label the nucleus to determine cellular localization and relative distributions of biotin-HDAP2 and other cellular probes. Fluorescence staining was observed with a Nikon Eclipse 50i microscope (20× objective). To determine mitochondrial localization of biotin-HDAP2, the same cells were also stained with 10 nM of MitoTracker red CMXRos, before fixation.

SA fluorescence was normalized to Hoechst fluorescence to account for differences in cell number in each field. The intensity of SA fluorescence was measured in each field, using the “Analyze” feature in ImageJ to compute the mean pixel intensity (in arbitrary fluorescence units) from the images. Differences among groups were compared by one-way ANOVA. Post hoc analyses were carried out using Tukey's multiple comparisons test.

Effects of peptides on cell survival in serum-free media: Cell survival in serum starvation conditions was studied in the presence and absence of biotin-HDAP2 using MDBK cells. Cells were plated at a density of 1-2×10⁵ cells/well in 96 well plates in DMEM/10% FBS media. The following day, cells were washed and the medium replaced with DMEM serum-free media in the presence or absence of different concentrations of biotin-HDAP2 and incubated for 5 days. Cell viability was assessed using the Resaruzin (Alamar blue) indicator dye. Quantitative analysis of dye conversion was measured using a fluorescent plate reader (ex/em=550/580 nm) and viability was expressed as fold increase over of untreated cells.

Effects of peptides on mitochondrial potential in the presence of Carbonyl cyanide m-chlorophenylhydrazone (CCCP): ARPE-19 cells (ATTC-CRL-2302) (5×10⁴ cells) were seeded in 35 mm glass dishes in DMEM/F12 medium with 10% FBS for 24 hr. On the day of the experiment, cells were cultured in DMEM/F12 in the presence of different concentrations of biotin-HDAP2 for 60 min (FIG. 17B). Then, cells were incubated with 10 μM CCCP in the presence of 5 nM TMRM, 100 nM Mito View Green, and 10 μg/ml Hoechst 33342 for 15 min at 37° C. Fluorescent images were immediately obtained with a Nikon Eclipse 50i fluorescence microscope (60× objective) using FITC (Mito View Green), Texas Red (TMRM), and DAPI (Hoechst) filters. Images were collected from three independent experiments, using 4-6 random fields for each treatment group, and imaged and analyzed as described for NR.

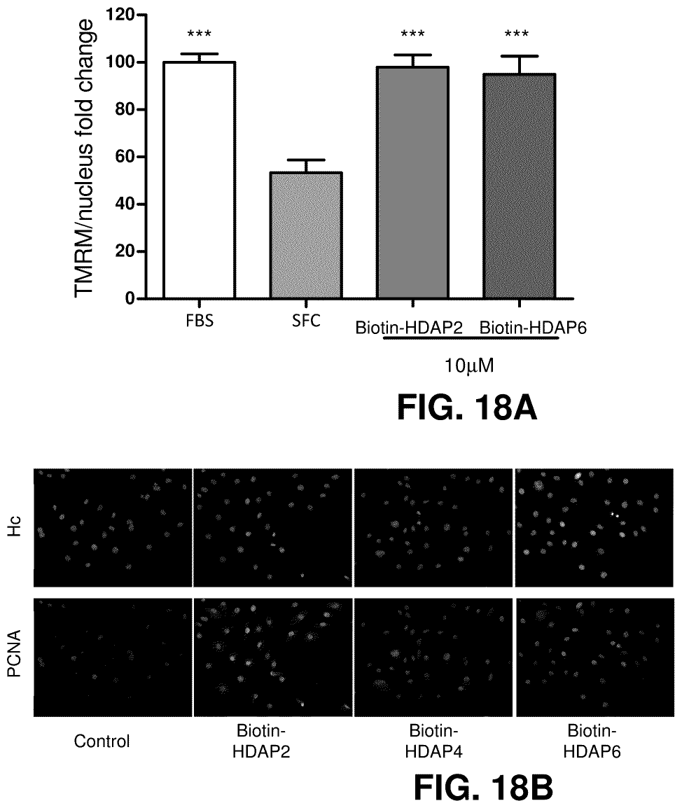

FIG. 18A shows the results of quantitative analysis of the effect of biotin-HDAP2 (SEQ ID NO: 1) and biotin-HDAP6 (SEQ ID NO: 10) on the restoration of mitochondrial membrane potential in ARPE-19 cells after 3 days of serum starvation. biotin-HDAP2 and biotin-HDAP6 were incubated with the cells for 2 hours after 3 days of serum starvation and mitochondrial membrane potential was measured using TMRM. Serum starvation for 3 days reduced mitochondrial membrane potential in serum-free controls (SFC), compared to regular cells grown on serum (FBS). Biotin-HDAP2 and biotin-HDAP6 restored mitochondrial membrane potential within 2 hours of incubation with the serum-starved cells, compared the serum-free controls (SFC). Error bars represent SEM (n=5) ***P<0.001.

FIG. 18B are representative images of HK-2 cells serum starved for 3 days in the presence or absence of biotin-HDAP2 (SEQ ID NO: 1), HDAP4 (SEQ ID NO: 13), and biotin-HDAP6 (SEQ ID NO: 10) and then tested for the expression of Proliferating Cell Nuclear Antigen (PCNA) (bottom row), a positive marker for cell proliferation. The Hoechst 33342 probe (Hc) was used to label the nucleus to determine cellular localization (top row). These images demonstrate that biotin-HDAP2, biotin-HDAP4, and biotin-HDAP6 increase proliferation expression of PCNA and cell proliferation during serum starvation, compared to control cells.

### Methods of Treating

The peptides are useful in treating any disease or condition that is associated with metabolic starvation. Such diseases and conditions include, but are not limited to, ischemia and/or reperfusion of a tissue or organ, hypoxia and any of a number of neurodegenerative diseases. Mammals in need of treatment or prevention of metabolic starvation are those mammals suffering from these diseases or conditions.

Ischemia in a tissue or organ of a mammal is a multifaceted pathological condition that is caused by oxygen deprivation (hypoxia) and/or glucose (e.g., substrate) deprivation. Oxygen and/or glucose deprivation in cells of a tissue or organ leads to a reduction or total loss of energy generating capacity and consequent loss of function of active ion transport across the cell membranes. Oxygen and/or glucose deprivation also leads to pathological changes in other cell membranes, including instability in the mitochondrial membranes. Thus, apoptotic proteins, normally compartmentalized within the mitochondria, may leak out into the cytoplasm and cause apoptotic cell death.

Ischemia or hypoxia in a particular tissue or organ may be caused by a loss or severe reduction in blood supply to the tissue or organ. The loss or severe reduction in blood supply may, for example, be due to thromboembolic stroke, coronary atherosclerosis, or peripheral vascular disease. The tissue affected by ischemia or hypoxia is typically muscle, such as cardiac, skeletal, or smooth muscle.

The organ affected by ischemia or hypoxia may be any organ that is subject to ischemia or hypoxia. Examples of organs affected by ischemia or hypoxia include brain, heart, kidney, and prostate. For instance, cardiac muscle ischemia or hypoxia is commonly caused by atherosclerotic or thrombotic blockages, which lead to the reduction or loss of oxygen delivery to the cardiac tissues by the cardiac arterial and capillary blood supply. Such cardiac ischemia or hypoxia may cause pain and necrosis of the affected cardiac muscle and ultimately may lead to cardiac failure.

Ischemia or hypoxia in skeletal muscle or smooth muscle may arise from similar causes. For example, ischemia or hypoxia in intestinal smooth muscle or skeletal muscle of the limbs may also be caused by atherosclerotic or thrombotic blockages.

Reperfusion is the restoration of blood flow to any organ or tissue in which the flow of blood is decreased or blocked. For example, blood flow can be restored to any organ or tissue affected by ischemia or hypoxia. The restoration of blood flow (reperfusion) can occur by any method known to those in the art. For instance, reperfusion of ischemic cardiac tissues may arise from angioplasty, coronary artery bypass graft, or the use of thrombolytic drugs.

The methods can also be used in the treatment or prophylaxis of neurodegenerative diseases associated with metabolic starvation. Neurodegenerative diseases associated with metabolic starvation include, for instance, Parkinson's disease, Alzheimer's disease, Huntington's disease and Amyotrophic Lateral Sclerosis (ALS, also known as Lou Gehrig's disease). The methods can be used to delay the onset or slow the progression of these and other neurodegenerative diseases associated with metabolic starvation such as glaucoma, macular degeneration, diabetic retinopathy and traumatic optic neuropathy. In one embodiment, the methods are used to treat optic nerve injury and axonal injury of retina ganglion cells, including traumatic optic nerve injury and glaucoma.

The analogs may help preserve mammalian organs prior to transplantation. For example, a removed organ can be susceptible to metabolic starvation due to lack of blood flow. Therefore, the peptides can be used to prevent metabolic starvation in the removed organ in the short period prior to transplantation. For example, a removed heart can be placed in a cardioplegic solution containing the analogs described above. The concentration of analogs in the standard buffered solution can be easily determined by those skilled in the art. Such concentrations may be, for example, between about 0.1 μM to about 10 μM. In one embodiment, the concentration is about 1 μM to about 10 μM.

The peptides may also be administered to a mammal taking a drug to treat a condition or disease. If a side effect of the drug includes metabolic starvation, mammals taking such drugs would greatly benefit from the disclosed peptides.

### Optic Nerve Protection

A series of experiments were conducted to determine if biotin-HDAP2 preserves mitochondrial ultrastructure in retinal ganglion cells (RGC) axons, whether biotin-HDAP2 preserves the integrity of axons in the optic nerve and/or whether biotin-HDAP2 treatment improves RGC survival. To these ends, 100 DBA/2J mice (8 and 12 weeks old) were obtained with the goal of studying them at 6 and 12 months of age. A small number of animals were examined at 6 months, but they had not yet developed elevated intraocular pressure (IOP), and no differences were observed between treatment conditions in optic nerve appearance or retinal ganglion cell distribution. Therefore, further analysis was delayed until animals reached 10 months, when elevated IOP was reliably present. These data provided a robust and reliable dataset with striking evidence that biotin-HDAP2 protects the optic nerve, mitochondria, and RGCs from IOP-related damage.

### Mitochondrial Ultrastructure in RGC Axons

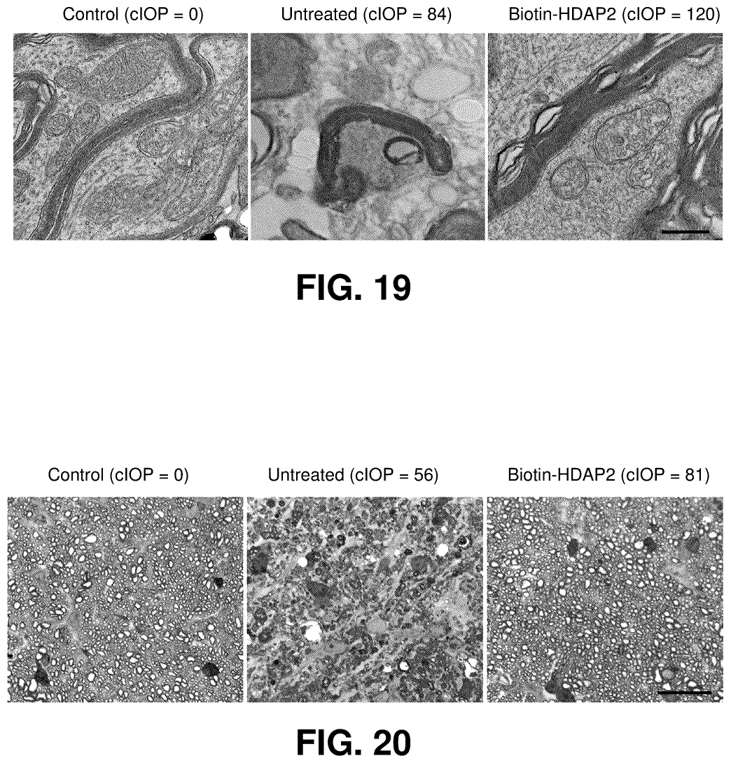

Optic nerves from all three experimental conditions (control, untreated and biotin-HDAP2-treated) have been embedded for ultrastructural analysis. Initial TEM observations of mitochondria in nerves exposed to high IOP show that mitochondria in biotin-HDAP2-treated animals are more numerous and exhibit preserved cristae and greater structural integrity compared to untreated controls (FIG. 19). Although quantitative analysis is ongoing, these qualitative differences strongly suggest a protective effect of biotin-HDAP2.

### Preservation of Optic Nerve Axons

Nerves were obtained from 10-month-old animals who had high IOP for at least 3 consecutive months. Differences between control, untreated and biotin-HDAP2-treated groups were striking and correlated closely with cumulative intraocular pressure (cIOP) exposure. On average, untreated nerves showed significant degeneration, whereas biotin-HDAP2-treated nerves were morphologically comparable to those of young controls (FIG. 20).

The representative sample already shows significant differences across groups (Kruskal-Wallis H-test (H=37.41, p=7.5×10⁹)), with post-hoc comparisons confirming that biotin-HDAP2 treatment significantly preserved axon structure. Moreover, biotin-HDAP2-treated nerves were morphologically similar to controls (FIG. 20).

### RGC Survival

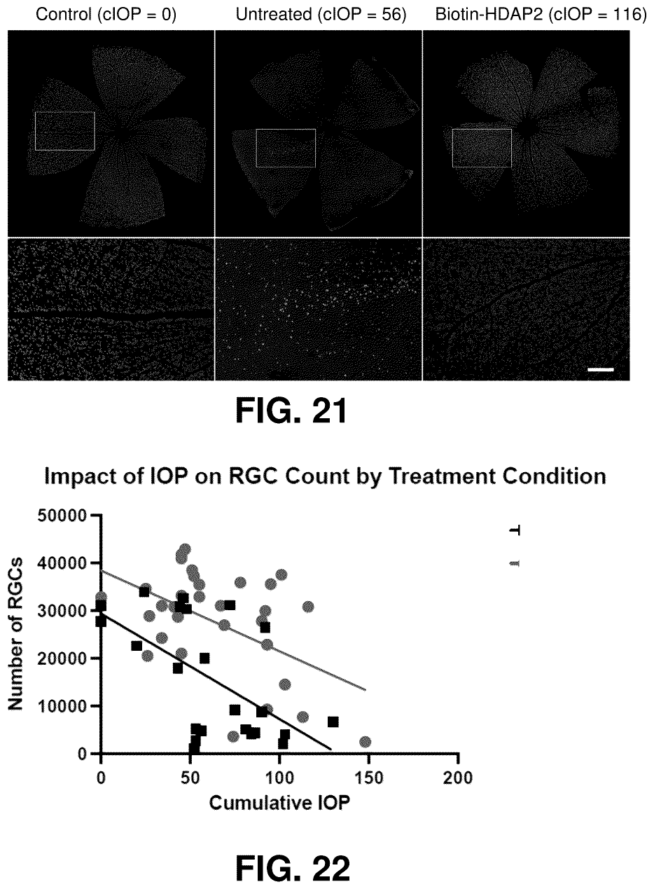

All retinas in this study were stained with RBPMS, imaged using confocal microscopy, and RGCs were automatically counted using ImageJ. To assess the overall effect of biotin-HDAP2 treatment on RGC survival, RGC counts between biotin-HDAP2-treated and Untreated animals were compared across all available time points. Biotin-HDAP2-treated animals exhibited significantly higher RGC counts (mean=26,267±14,440, n=23) compared to untreated controls (mean=13,336±11,645, n=16). Welch's t-test revealed that this difference was statistically significant (t=3.09, p=0.0039), supporting a robust neuroprotective effect of biotin-HDAP2 when evaluated across the full experimental cohort. See FIG. 21.

The effect of cIOP on RGC survival in untreated versus biotin-HDAP2-treated groups (FIG. 22) was also evaluated. In both, RGC counts declined with increasing cIOP, with a trend toward a steeper decline in biotin-HDAP2-treated animals. However, the difference in slopes was not significant (p=0.64), suggesting a similar rate of cell loss. Importantly, biotin-HDAP2-treated animals had significantly higher RGC counts overall, even after adjusting for IOP (F(1,35)=19.07, p<0.001), confirming a robust protective benefit across a range of pressure exposures. The data set includes both 10 and 12-month-old animals.

Referring to FIG. 23, the presence of biotin covalently bound to biotin-HDAP2 protected MDBK cells during serum starvation. Biotin-free HDAP2 did not provide protection. Specifically, treatment of MDBK cells with 1 μM of biotin-HDAP2 for 5 days during serum starvation increased AlamarBlue fluorescence in, indicating that the peptide promoted survival of MBDK cells. Treatment of cell with 1 μM of biotin-free HDAP2 did not increase AlamarBlue fluorescence, indicating that only biotinylated peptide (biotin-HDAP2) has structural characteristics to promote cell survival during stress. Error bars represent SEM (n=3) ***P<0.0001.

FIG. 24 is a graph demonstrating topical antimycin-induced scratching in mice is inhibited by biotin-HDAP2. However, antimycin-induced scratching in mice was potentiated by biotin-free HDAP2. Dermal administration of antimycin, a mitochondrial Complex III irreversible inhibitor, is known to induce ROS formation and oxidative stress in C fibers of sensory neurons, resulting in severe scratching in mice. Biotin-HDAP2 and biotin-free HDAP2 were tested to see if either can inhibit topical antimycin-induced itching in 12 months old C57BL/6J mice. Only biotin-HDAP2 inhibited ntimycin-induced scratching in mice, indicating that only biotinylated peptide (biotin-HDAP2) has structural characteristics to inhibit antimycin-induced oxidative stress in C-fibers and scratching behavior in mice. Specifically, 200 μM of topical antimycin in methanol induced itching, which was monitored for 60 min. Methanol alone did not induce any scratching in mice. Topical administration of 100 μM of biotin-HDAP2 with antimycin inhibited scratching. Error bars represent SEM (n=3) **P<0.001. Topical administration of 100 μM of biotin-free HDAP2 with antimycin increased scratching by 2.5 fold. Error bars represent SEM (n=3) ***P<0.0001.

### Modes of Administration

The peptide is administered to a mammal in an amount effective in reducing the number of cells undergoing, or preventing, metabolic starvation. The effective amount is determined during pre-clinical trials and clinical trials by methods familiar to physicians and clinicians.

An effective amount of a peptide (e.g. in a pharmaceutical composition) may be administered to a mammal in need thereof by any of a number of well-known methods for administering pharmaceutical compounds.

The analog may be administered systemically or locally. In one embodiment, the peptide is administered intravenously. In one embodiment, the administration is a constant rate intravenous infusion.

The analog can be injected directly into coronary artery during, for example, angioplasty or coronary bypass surgery, or applied onto coronary stents.

The analog may also be administered orally, topically, intranasally, intramuscularly, intraocularly, intraperitoneally, intravitreally, subcutaneously, or transdermally.

Other routes of administration include intracerebroventricularly or intrathecally. Intracerebroventricularly refers to administration into the ventricular system of the brain.

Any formulation known in the art of pharmacy is suitable for administration of the cationically-enframed high-density aromatic peptides. For oral administration, liquid or solid formulations may be used. Some examples of formulations include tablets, gelatin capsules, pills, troches, elixirs, suspensions, syrups, wafers, chewing gum and the like. The analog can be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

For systemic, intracerebroventricular, intrathecal, topical, intranasal, subcutaneous, or transdermal administration, formulations of the biotin-cationically-enframed high density aromatic peptides may utilize conventional diluents, carriers, or excipients etc., such as are known in the art can be employed to deliver the peptides. For example, the formulations may comprise one or more of the following: a stabilizer, a surfactant, (e.g. a nonionic surfactant), and optionally a salt and/or a buffering agent. The peptide may be delivered in the form of an aqueous solution, or in a lyophilized form.

The stabilizer may, for example, be an amino acid, such as for instance, glycine; or an oligosaccharide, such as for example, sucrose, tetralose, lactose or a dextran. Alternatively, the stabilizer may be a sugar alcohol, such as for instance, mannitol; or a combination thereof. In one embodiment the stabilizer or combination of stabilizers constitutes from about 0.1% to about 10% weight for weight of the peptide.

In one embodiment, the surfactant is a nonionic surfactant, such as a polysorbate. Some examples of suitable surfactants include Tween20, Tween80; a polyethylene glycol or a polyoxyethylene polyoxypropylene glycol, such as Pluronic F-68 at from about 0.001% (w/v) to about 10% (w/v).

The salt or buffering agent may be any salt or buffering agent, such as for example, sodium chloride, or sodium/potassium phosphate, respectively. In one embodiment, the buffering agent maintains the pH of the pharmaceutical composition in the range of about 5.5 to about 7.5. The salt and/or buffering agent is also useful to maintain the osmolality at a level suitable for administration to a human or an animal. In one embodiment, the salt or buffering agent is present at a roughly isotonic concentration of about 150 mM to about 300 mM.

The formulations of the peptides may additionally contain one or more conventional additives. Some examples of such additives include a solubilizer such as, for example, glycerol; an antioxidant such as for example, benzalkonium chloride (a mixture of quaternary ammonium compounds, known as “quats”), benzyl alcohol, chloretone or chlorobutanol; anesthetic agents such as, for example, a morphine derivative; or an isotonic agent etc., such as described above. As a further precaution against oxidation or other spoilage, the pharmaceutical compositions may be stored under nitrogen gas in vials sealed with impermeable stoppers.

The mammal can be any mammal, including, for example, farm animals, such as sheep, pigs, cows, and horses; pet animals, such as dogs and cats; laboratory animals, such as rats, mice and rabbits. In one embodiment, the mammal is a human.

Statistics: Mean values for each treatment and eccentricity were compared and tested for significance using either a Student's t-test or one-way analysis of variance (ANOVA) with graphing and statistical software (GraphPad Prism, GraphPad Software). P values less than 0.05 were considered significant and all data are expressed as means±SEM.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for treating an eye, the method comprising:
administering to an eye of a patient a composition of matter comprising biotin covalently bound to a peptide of dArg-Phe-Phe-dArg (SEQ ID NO: 1).

2. The method as recited in claim 1, wherein the composition of matter consists of the biotin and the peptide and water.

3. The method as recited in claim 1, wherein the peptide is formulated as an aqueous solution that is administered directly to the eye during the administering.

4. The method as recited in claim 1, wherein the patient is in need of treatment for a mitochondria dysfunction-associated disease.

5. The method as recited in claim 1, wherein the patient is in need of treatment for optic nerve injury.

6. The method as recited in claim 1, wherein the patient is in need of treatment for axonal injury of retina ganglion cells.

7. The method as recited in claim 1, wherein the patient is in need of treatment for glaucoma.

8. The method as recited in claim 1, wherein the administering administers the composition of matter to a retina of the eye.

9. A method for treating an eye, the method comprising: administering to an eye of a patient a peptide comprising:
an N-terminus and a C-terminus;
r amino acid residues, wherein 4≤r≤10 and the amino acid residues are either cationic amino acid residues or aromatic amino acid residues, the r amino acid residues including (1) a cationic D-amino acid (dC) at the N-terminus and (2) a cationic D-amino acid (mdC) at the C-terminus;
biotin covalently bound to the peptide;
wherein a net number of positive residues (np), including the cationic D-amino acid (dC) and the cationic D-amino acid (mdC), is 2≤np≤r−2; and
wherein a net number of the aromatic amino acid residues (na) is na≥np, and the aromatic amino acid residues include at least two aromatic amino acids that are sequentially positioned in the peptide.

10. The method as recited in claim 9, wherein the aromatic amino acid residues are selected from a group consisting of phenylalanine, tyrosine, tryptophan and combinations thereof.

11. The method as recited in claim 9, wherein the peptide is selected from the group consisting of: dArg-Phe-Phe-dArg (SEQ ID NO: 1), dArg-Trp-Trp-dLys (SEQ ID NO: 10, HDAP6) and dArg-Phe-Phe-Phe-dArg (SEQ ID NO: 13, HDAP4).

12. The method as recited in claim 9, wherein the cationic amino acid residues are selected from arginine, histidine, lysine and combinations thereof.

13. The method as recited in claim 9, wherein the cationic amino acid residues are selected from D-arginine, D-histidine, D-lysine and combinations thereof.

14. The method as recited in claim 9, wherein 4≤r≤6.

15. The method as recited in claim 9, wherein r is 4.

16. The method as recited in claim 9, wherein the aromatic amino acid residues are selected from phenylalanine, tyrosine, tryptophan and combinations thereof and the cationic amino acid residues are selected from D-arginine, D-histidine, D-lysine and combinations thereof, the aromatic amino acid residues having at least two sequential aromatic amino acid residues.

17. The method as recited in claim 16, wherein 4≤r≤8.

18. The method as recited in claim 16, wherein 4≤r≤6.

19. The method as recited in claim 16, wherein r is 4.

20. The method as recited in claim 9, wherein the peptide is formulated as an aqueous solution that is administered to the eye during the administering.

21. The method as recited in claim 20, wherein the at least two aromatic amino acids that are the same amino acid, 4≤r≤6, the aromatic amino acid residues are selected from phenylalanine, tyrosine, tryptophan and combinations thereof and the cationic amino acid residues are selected from D-arginine, D-histidine, D-lysine and combinations thereof.

22. The method as recited in claim 21, wherein the aromatic amino acid residues are selected from phenylalanine, tyrosine, tryptophan and combinations thereof and the cationic amino acid residues are selected from D-arginine, D-histidine, D-lysine and combinations thereof, the aromatic amino acid residues having at least two sequential aromatic amino acid residues.

23. The method as recited in claim 9, wherein the aromatic amino acid residues include at least three aromatic amino acids that are sequentially positioned in the peptide.

24. The method as recited in claim 23, wherein the at least three aromatic amino acids are selected from a group consisting of phenylalanine, tyrosine, tryptophan and combinations thereof.

25. The method as recited in claim 23, wherein the at least three aromatic amino acids are the same amino acid.


SEQUENCE LISTING (24 sequences)

SEQ ID NO: 1 - protein, 4 aa, synthetic construct
  SITE 1: note = D-arginine
  SITE 4: note = D-arginine
RFFR

SEQ ID NO: 2 - protein, 4 aa, Synthetic construct
  SITE 1: note = D-arginine
  SITE 4: note = D-lysine
RFFK

SEQ ID NO: 3 - protein, 4 aa, synthetic construct
  SITE 1: note = D-lysine
  SITE 4: note = D-arginine
KFFR

SEQ ID NO: 4 - protein, 4 aa, synthetic construct
  SITE 1: note = D-lysine
  SITE 4: note = D-lysine
KFFK

SEQ ID NO: 5 - protein, 4 aa, synthetic construct
  SITE 1: note = D-arginine
  SITE 4: note = D-arginine
RYYR

SEQ ID NO: 6 - protein, 4 aa, synthetic construct
  SITE 1: note = D-arginine
  SITE 4: note = D-lysine
RYYK

SEQ ID NO: 7 - protein, 4 aa, synthetic construct
  SITE 1: note = D-lysine
  SITE 4: note = D-arginine
KYYR

SEQ ID NO: 8 - protein, 4 aa, synthetic construct
  SITE 1: note = D-lysine
  SITE 4: note = D-lysine
KYYK

SEQ ID NO: 9 - protein, 4 aa, synthetic construct
  SITE 1: note = D-arginine
  SITE 4: note = D-arginine
RWWR

SEQ ID NO: 10 - protein, 4 aa, synthetic construct
  SITE 1: note = D-arginine
  SITE 4: note = D-lysine
RWWK

SEQ ID NO: 11 - protein, 4 aa, synthetic construct
  SITE 1: note = D-lysine
  SITE 4: note = D-arginine
KWWR

SEQ ID NO: 12 - protein, 4 aa, synthetic construct
  SITE 1: note = D-lysine
  SITE 4: note = D-lysine
KWWK

SEQ ID NO: 13 - protein, 5 aa, synthetic construct
  SITE 1: note = D-arginine
  SITE 5: note = D-arginine
RFFFR

SEQ ID NO: 14 - protein, 5 aa, synthetic construct
  SITE 1: note = D-arginine
  SITE 5: note = D-lysine
RFFFK

SEQ ID NO: 15 - protein, 5 aa, synthetic construct
  SITE 1: note = D-lysine
  SITE 5: note = D-arginine
KFFFR

SEQ ID NO: 16 - protein, 5 aa, synthetic construct
  SITE 1: note = D-lysine
  SITE 5: note = D-lysine
KFFFK

SEQ ID NO: 17 - protein, 5 aa, synthetic construct
  SITE 1: note = D-arginine
  SITE 5: note = D-arginine
RYYYR

SEQ ID NO: 18 - protein, 5 aa, synthetic construct
  SITE 1: note = D-arginine
  SITE 5: note = D-lysine
RYYYK

SEQ ID NO: 19 - protein, 5 aa, synthetic construct
  SITE 1: note = D-lysine
  SITE 5: note = D-arginine
KYYYR

SEQ ID NO: 20 - protein, 5 aa, synthetic construct
  SITE 1: note = D-lysine
  SITE 5: note = D-lysine
KYYYK

SEQ ID NO: 21 - protein, 5 aa, synthetic construct
  SITE 1: note = D-arginine
  SITE 5: note = D-arginine
RWWWR

SEQ ID NO: 22 - protein, 5 aa, synthetic construct
  SITE 1: note = D-arginine
  SITE 5: note = D-lysine
RWWWK

SEQ ID NO: 23 - protein, 5 aa, synthetic construct
  SITE 1: note = D-lysine
  SITE 5: note = D-arginine
KWWWR

SEQ ID NO: 24 - protein, 5 aa, synthetic construct
  SITE 1: note = D-lysine
  SITE 5: note = D-lysine
KWWWK